(19) United States
(12) Patent Application Publication
(10) Pub. No.: US 2019/0328290 A1
(43) Pub. Date: Oct. 31, 2019

(54) GLUCOSE SENSOR-BASED TRACKING SYSTEM

(71) Applicant: Medtronic MiniMed, Inc., Northbridge, CA (US)

(72) Inventors: Louis Dias, Santa Monica, CA (US); Zachary J. Finley, Moorpark, CA (US); Robert T. Manning, Dunwoody, GA (US); Auriana Ojeda, Pasadena, CA (US); Hooman C. Hakami, River Hills, WI (US); Jeffrey L. Anglin, Agoura Hills, CA (US)

(21) Appl. No.: 16/379,827

(22) Filed: Apr. 10, 2019

Related U.S. Application Data
(60) Provisional application No. 62/663,351, filed on Apr. 27, 2018.
(60) Provisional application No. 62/663,464, filed on Apr. 27, 2018.

(51) Int. Cl. A61B 5/145; A61B 5/00

(52) U.S. Cl. CPC A61B 5/14532; A61B 5/486; A61B 5/0022

(57) ABSTRACT

A method includes providing access to assets through communication channels, to a community of users associated with a medical condition, where access is provided by a system including a communication control that selectively enables user devices. Goals are established to modify a health-related outcome of the community of users over time and effect a particular treatment for the medical condition. The goals can include a time-in-range metric that defines an amount of time that a user of the community of users maintains the health-related outcome within a range bounded by a lower limit and an upper limit. The goals can include a total days of sensor data metric that defines an amount of time that the user wore a glucose sensor. Access to engagement rewards can be enabled based on the interactions and/or achievement of the goals to improve one or more health outcomes associated with the medical condition.

### CROSS-REFERENCE TO RELATED APPLICATIONS

This application claims the benefit of priority to U.S. Provisional Application No. 62/663,351 filed Apr. 27, 2018 and claims the benefit of priority to U.S. Provisional Application No. 62/663,464 filed Apr. 27, 2018, the disclosures of which are incorporated herein by reference in their entirety.

### FIELD

The present technology is generally related to data management, and more particularly to a glucose sensor-based tracking system for medical condition control.

### BACKGROUND

Individuals with chronic medical conditions may rely upon a number of devices, systems, and expert care to manage their conditions. Chronic medical conditions, such as diabetes, can require regular monitoring to observe condition metrics, such as blood sugar level, and provide corresponding medication dosages in response thereto. Personal choices made by individuals with chronic medical conditions can also impact medication dosage, delivery rate, and frequency of dosage adjustments needed to manage the effects of the chronic medical conditions. For example, dietary intake, exercise, rest, and other factors can substantially impact medical equipment usage patterns and medication requirements.

### SUMMARY

This disclosure generally relates to systems, methods, and computer program products for medical condition control. In one aspect, the present disclosure provides an engagement system for a targeted user with diabetes. The engagement system can include at least one computer to enable interactions with the targeted user. The engagement system can increase effectiveness of a diabetes therapy and effect a particular treatment for diabetes for the targeted user by suggesting actions or rewarding the targeted user for taking actions to improve diabetes health outcomes measured by at least one diabetes health outcome metric that meets a success criteria in response to the interactions or taken actions by the targeted user. The engagement system can also include a server communicatively coupled with the at least one computer. The server can include a data storage system with a processing system configured to perform a plurality of operations. The operations can include receiving an upload of sensed glucose data from a glucose monitor associated with the targeted user and collected over a first time period. The sensed glucose data can be continuous sensor data from a monitored continuous sensor that provides sensed glucose data at least once every 20 minutes over at least 12 hours or includes on demand uploads of the continuous sensor data from the targeted user. The sensed glucose data associated with the targeted user can be stored in the data storage system along with other uploads of sensed glucose data associated with the targeted user. At least a subset of the stored sensed glucose data over a second time period can be extracted from the data storage system. The at least one diabetes health outcome metric can be computed based on the stored sensed glucose sensor data associated with the targeted user over the second time period. The computed at least one diabetes health outcome metric based on the stored sensed glucose sensor data associated with the targeted user can be transferred to the at least one computer. The at least one computer can interact with the targeted user based on the transferred at least one diabetes health outcome metric with further interactions, suggested actions or rewards provided to the targeted user based on whether the computed at least one diabetes health outcome metric met the success criteria. The computed at least one diabetes health outcome metric can be a time-in-range for the subset of the stored sensed glucose data associated with the targeted user, and the time-in-range can be the time that the subset of the stored sensed glucose data is not in a low sensor glucose state.

In addition to one or more of the features described above or below, or as an alternative, further embodiments may include where the interactions on the at least one computer with the targeted user include at least one of: asking questions, logging results, providing articles related to the diabetes therapy, providing glucose sensor data graphs, providing analysis of glucose sensor data, providing recommendations of diabetes therapy suggested actions, displaying the computed at least one diabetes health outcome metric, notifying the targeted user on whether success criteria of the computed at least one diabetes health outcome metric has been met, and/or notifying the targeted user of a reward.

In addition to one or more of the features described above or below, or as an alternative, further embodiments may include where the low sensor glucose state is below 60 mg/dl.

In addition to one or more of the features described above or below, or as an alternative, further embodiments may include where the time-in-range is the time that the subset of the stored sensed glucose data is also not in a high sensor glucose state.

In addition to one or more of the features described above or below, or as an alternative, further embodiments may include where the high sensor glucose state is above 180 mg/dl.

In addition to one or more of the features described above or below, or as an alternative, further embodiments may include where the time-in-range is shown as a percent of the second time period.

In addition to one or more of the features described above or below, or as an alternative, further embodiments may include where the success criteria for the time-in-range is at or above 75%.

In addition to one or more of the features described above or below, or as an alternative, further embodiments may include where the success criteria for the time-in-range is at or above 80%.

In addition to one or more of the features described above or below, or as an alternative, further embodiments may include where the low sensor glucose state is below 60 mg/dl and the high sensor glucose state is above 180 mg/dl.

In addition to one or more of the features described above or below, or as an alternative, further embodiments may include where the rewards include at least one of: points, points redeemable for merchandise, a credit on an account, a credit applied to a co-pay, and/or an achievement badge.

In addition to one or more of the features described above or below, or as an alternative, further embodiments may include where the at least one computer is selected from the group including a personal computer, laptop computer, a cloud computing resource, or an ultra-book computer.

In addition to one or more of the features described above or below, or as an alternative, further embodiments may include where the at least one computer is selected from the group including a cellular telephone, a smartphone, a wearable computing device, a personal digital assistant, or a tablet.

In another aspect, the present disclosure provides a method that can include receiving, by a server communicatively coupled with at least one computer, an upload of sensed glucose data from a glucose monitor associated with a targeted user with diabetes and collected over a first time period. The sensed glucose data associated with the targeted user can be stored in a data storage system of the server along with other uploads of sensed glucose data associated with the targeted user. The sensed glucose data can be continuous sensor data from a monitored continuous sensor that provides sensed glucose data at least once every 20 minutes over at least 12 hours or includes on demand uploads of the continuous sensor data from the targeted user. At least a subset of the stored sensed glucose data over a second time period can be extracted from the data storage system. At least one diabetes health outcome metric can be computed based on the stored sensed glucose sensor data associated with the targeted user over the second time period. The computed at least one diabetes health outcome metric can be transferred based on the stored sensed glucose sensor data associated with the targeted user to the at least one computer. The at least one computer can interact with the targeted user based on the transferred at least one diabetes health outcome metric with interactions, suggested actions or rewards to the targeted user based on whether the computed at least one diabetes health outcome metric met a success criteria to increase effectiveness of a diabetes therapy. A particular treatment for diabetes can be affected for the targeted user by at least suggesting actions or rewarding the targeted user for taking actions to improve diabetes health outcomes measured by the computed at least one diabetes health outcome metric that meets the success criteria in response to the interactions or taken actions by the targeted user. The computed at least one diabetes health outcome metric can be a time-in-range for the subset of the stored sensed glucose data associated with the targeted user, and the time-in-range can be the time that the subset of the stored sensed glucose data is not in a low sensor glucose state.

In addition to one or more of the features described above or below, or as an alternative, further embodiments may include where the time-in-range is the time that the subset of the stored sensed glucose data is not in a low sensor glucose state below 60 mg/dl and the time-in-range is the time that the subset of the stored sensed glucose data is also not in a high sensor glucose state above 180 mg/dl.

In addition to one or more of the features described above or below, or as an alternative, further embodiments may include where the time-in-range is shown as a percent of the second time period and the success criteria for the time-in-range is at or above 75%.

In another aspect, the present disclosure provides a computer program product including a storage medium embodied with computer program instructions that when executed by a server communicatively coupled with at least one computer cause the server to implement a plurality of operations. The operations can include receiving an upload of sensed glucose data from a glucose monitor associated with a targeted user with diabetes and collected over a first time period, where the sensed glucose data can be continuous sensor data from a monitored continuous sensor that provides sensed glucose data at least once every 20 minutes over at least 12 hours or includes on demand uploads of the continuous sensor data from the targeted user. The operations can also include storing the sensed glucose data associated with the targeted user in a data storage system of the server along with other uploads of sensed glucose data associated with the targeted user. The operations can further include extracting at least a subset of the stored sensed glucose data over a second time period from the data storage system and computing at least one diabetes health outcome metric based on the stored sensed glucose sensor data associated with the targeted user over the second time period. The operations can also include transferring the computed at least one diabetes health outcome metric based on the stored sensed glucose sensor data associated with the targeted user to the at least one computer. The at least one computer can interact with the targeted user based on the transferred at least one diabetes health outcome metric with interactions, suggested actions or rewards to the targeted user based on whether the computed at least one diabetes health outcome metric met a success criteria to increase effectiveness of a diabetes therapy. A particular treatment for diabetes can be affected for the targeted user by suggesting actions or rewarding the targeted user for taking actions to improve diabetes health outcomes measured by the computed at least one diabetes health outcome metric that meets the success criteria in response to the interactions or taken actions by the targeted user. The computed at least one diabetes health outcome metric can be a time-in-range for the subset of the stored sensed glucose data associated with the targeted user, and the time-in-range can be the time that the subset of the stored sensed glucose data is not in a low sensor glucose state.

In one aspect, the present disclosure provides an engagement system for a targeted user with diabetes. The engagement system can include at least one computer to enable interactions with the targeted user. The engagement system can increase effectiveness of a diabetes therapy and effect a particular treatment for diabetes for the targeted user by suggesting actions or rewarding the targeted user for taking actions to improve diabetes health outcomes measured by at least one diabetes health outcome metric that meets a success criteria in response to the interactions or taken actions by the targeted user. The engagement system can also include a server communicatively coupled with the at least one computer. The server can include a data storage system with a processing system configured to perform a plurality of operations. The operations can include receiving an upload of medical device mode data associated with the targeted user and collected over a first time period, and where the medical device mode data can be for a device to control diabetes in a closed-loop mode for at least a portion of the first time period. The medical device mode data associated with the targeted user can be stored in the data storage system along with other uploads of medical device mode data associated with the targeted user. At least a subset of the stored medical device mode data over a second time period can be extracted from the data storage system. The at least one diabetes health outcome metric can be computed based on the stored medical device mode data associated with the targeted user over the second time period. The computed at least one diabetes health outcome metric based on the stored medical device mode data associated with the targeted user can be transferred to the at least one computer. The at least one computer can interact with the targeted user based on the transferred at least one diabetes health outcome metric with further interactions, suggested actions or rewards provided to the targeted user based on whether the computed at least one diabetes health outcome metric met the success criteria. The computed at least one diabetes health outcome metric can be a time-in-closed-loop mode for the subset of stored medical device mode data associated with the targeted user, and the time-in-closed-loop mode can be the time that the subset of the stored medical device mode data is not in open-loop mode.

In addition to one or more of the features described above or below, or as an alternative, further embodiments may include where the interactions on the at least one computer with the targeted user include at least one of: asking questions, logging results, providing articles related to the diabetes therapy, providing medical device mode data graphs, providing analysis of medical device mode data, providing recommendations of diabetes therapy suggested actions, displaying the computed at least one diabetes health outcome metric, notifying the targeted user on whether success criteria of the computed at least one diabetes health outcome metric has been met, or notifying the targeted user of an reward.

In addition to one or more of the features described above or below, or as an alternative, further embodiments may include where the time-in-closed-loop mode is based on data obtained from a system including an insulin infusion device and a glucose sensor in communication with the insulin infusion device.

In addition to one or more of the features described above or below, or as an alternative, further embodiments may include where the time-in-closed-loop mode is shown as a percent of the second time period.

In addition to one or more of the features described above or below, or as an alternative, further embodiments may include where the success criteria for the time-in-closed-loop mode is at or above 75%.

In addition to one or more of the features described above or below, or as an alternative, further embodiments may include where the success criteria for the time-in-closed-loop mode is at or above 80%.

In addition to one or more of the features described above or below, or as an alternative, further embodiments may include where the rewards include at least one of: points, points redeemable for merchandise, a credit on an account, a credit applied to a co-pay, and/or an achievement badge.

In addition to one or more of the features described above or below, or as an alternative, further embodiments may include where the at least one computer is selected from the group including a personal computer, laptop computer, a cloud computing resource, or an ultra-book computer.

In addition to one or more of the features described above or below, or as an alternative, further embodiments may include where the at least one computer is selected from the group including a cellular telephone, a smartphone, a wearable computing device, a personal digital assistant, or a tablet.

In another aspect, the present disclosure provides a method that can include receiving, by a server communicatively coupled with at least one computer, an upload of medical device mode data associated with a targeted user with diabetes and collected over a first time period. The medical device mode data associated with the targeted user can be stored in a data storage system of the server along with other uploads of medical device mode data associated with the targeted user. The medical device mode data can be for a device to control diabetes in a closed-loop mode for at least a portion of the first time period. At least a subset of the stored medical device mode data over a second time period can be extracted from the data storage system. At least one diabetes health outcome metric can be computed based on the stored medical device mode data associated with the targeted user over the second time period. The computed at least one diabetes health outcome metric can be transferred based on the stored medical device mode data associated with the targeted user to the at least one computer. The at least one computer can interact with the targeted user based on the transferred at least one diabetes health outcome metric with interactions, suggested actions or rewards to the targeted user based on whether the computed at least one diabetes health outcome metric met a success criteria to increase effectiveness of a diabetes therapy. A particular treatment for diabetes can be affected for the targeted user by at least suggesting actions or rewarding the targeted user for taking actions to improve diabetes health outcomes measured by the computed at least one diabetes health outcome metric that meets the success criteria in response to the interactions or taken actions by the targeted user. The computed at least one diabetes health outcome metric can be a time-in-closed-loop mode for the subset of stored medical device mode data associated with the targeted user, and the time-in-closed-loop mode can be the time that the subset of the stored medical device mode data is not in open-loop mode.

In addition to one or more of the features described above or below, or as an alternative, further embodiments may include where the interactions on the at least one computer with the targeted user include at least one of: asking questions, logging results, providing articles related to the diabetes therapy, providing medical device mode data graphs, providing analysis of medical device mode data, providing recommendations of diabetes therapy suggested actions, displaying the computed at least one diabetes health outcome metric, notifying the targeted user on whether success criteria of the computed at least one diabetes health outcome metric has been met, and/or notifying the targeted user of an reward.

In another aspect, the present disclosure provides a computer program product including a storage medium embodied with computer program instructions that when executed by a server communicatively coupled with at least one computer cause the server to implement a plurality of operations. The operations can include receiving an upload of medical device mode data associated with a targeted user with diabetes and collected over a first time period. The medical device mode data can be for a device to control diabetes in a closed-loop mode for at least a portion of the first time period. The operations can also include storing the medical device mode data associated with the targeted user in a data storage system of the server along with other uploads of medical device mode data associated with the targeted user. The operations can further include extracting at least a subset of the stored medical device mode data over a second time period from the data storage system and computing at least one diabetes health outcome metric based on the stored medical device mode data associated with the targeted user over the second time period. The operations can also include transferring the computed at least one diabetes health outcome metric based on the stored medical device mode data associated with the targeted user to the at least one computer. The at least one computer can interact with the targeted user based on the transferred at least one diabetes health outcome metric with interactions, suggested actions or rewards to the targeted user based on whether the computed at least one diabetes health outcome metric met a success criteria to increase effectiveness of a diabetes therapy. A particular treatment for diabetes can be affected for the targeted user by suggesting actions or rewarding the targeted user for taking actions to improve diabetes health outcomes measured by the computed at least one diabetes health outcome metric that meets the success criteria in response to the interactions or taken actions by the targeted user. The computed at least one diabetes health outcome metric can be a time-in-closed-loop mode for the subset of stored medical device mode data associated with the targeted user, and the time-in-closed-loop mode can be the time that the subset of the stored medical device mode data is not in open-loop mode.

In one aspect, the present disclosure provides a method that includes providing access to a plurality of assets through a plurality of communication channels, to a community of users associated with a medical condition, where the access is provided by a system including a communication control that can selectively enable a plurality of user devices of the community of users in order to access the assets. One or more goals are established to modify a health-related outcome of the community of users over a period of time and effect a particular treatment for the medical condition. The one or more goals can include a time-in-range metric that defines an amount of time that a user of the community of users maintains the health-related outcome within a range bounded by a lower limit and an upper limit. The one or more goals can include a total days of sensor data metric that defines an amount of time that the user wore a glucose sensor. The total days of sensor data metric can be from continuous sensor data from a monitored continuous sensor that provides sensed glucose data at least once every 20 minutes over at least 12 hours or includes on demand uploads of the continuous sensor data from the user. Tracking of a plurality of interactions of the community of users with the assets and the progress towards achieving the one or more goals can be performed. Access to one or more engagement rewards can be enabled based on the interactions and/or achievement of the one or more goals to improve one or more health outcomes associated with the medical condition.

In addition to one or more of the features described above or below, or as an alternative, further embodiments may include where the assets include one or more embedded assets locally accessible by a computer system and one or more linked assets accessible through a network operably coupled to the computer system.

In addition to one or more of the features described above or below, or as an alternative, further embodiments may include varying the one or more goals to establish a plurality of challenges to the community of users, where the challenges are modified over time to increase a number of users trending towards achieving the health-related outcome.

In addition to one or more of the features described above or below, or as an alternative, further embodiments may include applying one or more models to sequence a plurality of activities, feedback, and engagement reward points in the challenges that encourage the community of users to achieve higher levels of a plurality of health tiers.

In addition to one or more of the features described above or below, or as an alternative, further embodiments may include where the activities include one or more of: data updates, educational resource access, surveys, video content, medication delivery scheduling, social media access, and interactions between the community of users.

In addition to one or more of the features described above or below, or as an alternative, further embodiments may include making one or more personalized adjustments to the challenges for one or more users of the community of users, and enabling one or more interactions between a plurality of users and/or coaches through the communication channels targeting one or more health outcome improvements.

In another aspect, the present disclosure provides a data storage system and a computer system communicatively coupled to the data storage system. The computer system includes a processing system configured to perform a plurality of operations. The operations can include providing access to a plurality of assets through a plurality of communication channels, to a community of users associated with a medical condition, where the access is provided through a communication control of the system that selectively enables a plurality of user devices of the community of users in order to access the assets. The operations can also include establishing one or more goals to modify a health outcome of the community of users over a period of time, and effecting a particular treatment for the medical condition. The one or more goals can include a time-in-range metric that defines an amount of time that a user of the community of users maintains the health-related outcome within a range bounded by a lower limit and an upper limit. The one or more goals can include a total days of sensor data metric that defines an amount of time that the user wore a glucose sensor. The total days of sensor data metric can be from continuous sensor data from a monitored continuous sensor that provides sensed glucose data at least once every 20 minutes over at least 12 hours or includes on demand uploads of the continuous sensor data from the user. The operations can additionally include tracking a plurality of interactions of the community of users with the assets and the progress towards achieving the one or more goals, and enabling access to one or more engagement rewards based on the interactions and/or achievement of the one or more goals to improve one or more health outcomes associated with the medical condition.

In addition to one or more of the features described above or below, or as an alternative, further embodiments may include where the assets include one or more embedded assets locally accessible by the computer system and one or more linked assets accessible through a network operably coupled to the computer system.

In addition to one or more of the features described above or below, or as an alternative, further embodiments may include where the computer system is further configured to perform operations including varying the one or more goals to establish a plurality of challenges to the community of users, where the challenges are modified over time to increase a number of users trending towards achieving the health-related outcome, and applying one or more models to sequence a plurality of activities, feedback, and engagement reward points in the challenges that encourage the community of users to achieve higher levels of a plurality of health tiers, where the activities include one or more of: data updates, educational resource access, surveys, video content, medication delivery scheduling, social media access, and interactions between the community of users.

In addition to one or more of the features described above or below, or as an alternative, further embodiments may include where the computer system is further configured to perform operations including making one or more personalized adjustments to the challenges for one or more users of the community of users, and enabling one or more interactions between a plurality of users and/or coaches through the communication channels targeting one or more health outcome improvements.

In another aspect, the present disclosure provides a computer program product including a storage medium embodied with computer program instructions that when executed by a computer system cause the computer system to implement providing access to a plurality of assets through a plurality of communication channels, to a community of users associated with a medical condition, where the access is provided through a communication control of the computer system that selectively enables a plurality of user devices of the community of users in order to access the assets. One or more goals are established to modify a health outcome of the community of users over a period of time, and effecting a particular treatment for the medical condition. The one or more goals can include a time-in-range metric that defines an amount of time that a user of the community of users maintains the health-related outcome within a range bounded by a lower limit and an upper limit. The one or more goals can include a total days of sensor data metric that defines an amount of time that the user wore a glucose sensor. The total days of sensor data metric can be from continuous sensor data from a monitored continuous sensor that provides sensed glucose data at least once every 20 minutes over at least 12 hours or includes on demand uploads of the continuous sensor data from the user. Tracking of a plurality of interactions of the community of users with the assets and the progress towards achieving the one or more goals can be performed. Access to one or more engagement rewards can be enabled based on the interactions and/or achievement of the one or more goals to improve one or more health outcomes associated with the medical condition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a block diagram that illustrates a system for medical condition control according to an embodiment;

FIG. 2 is a block diagram that illustrates a user interface that can be displayed by the system of FIG. 1 according to an embodiment;

FIG. 3 is a diagram that illustrates a user summary that can be displayed as part of the user interface of FIG. 2 according to an embodiment;

FIG. 4 is a diagram that illustrates a current point balance that can be displayed as part of the user interface of FIG. 2 according to an embodiment;

FIG. 5 is a block diagram that illustrates another user interface that can be displayed by the system of FIG. 1 according to an embodiment;

FIG. 6 is a diagram that illustrates a time-based goal that can be displayed as part of the user interface of FIG. 5 according to an embodiment;

FIG. 7 is a diagram that illustrates result tracking for a time period that meets a time-based goal and can be displayed as part of the user interface of FIG. 5 according to an embodiment;

FIG. 8 is a diagram that illustrates result tracking for a time period that does not meet a time-based goal and can be displayed as part of the user interface of FIG. 5 according to an embodiment;

FIG. 9 is a diagram that illustrates result tracking for a not yet completed time period and can be displayed as part of the user interface of FIG. 5 according to an embodiment;

FIG. 10 is a block diagram that illustrates another user interface that can be displayed by the system of FIG. 1 according to an embodiment;

FIG. 11 is a diagram that illustrates a time-based goal and can be displayed as part of the user interface of FIG. 10 according to an embodiment;

FIG. 12 is a diagram that illustrates result tracking for a time period that meets a time-based goal and can be displayed as part of the user interface of FIG. 10 according to an embodiment;

FIG. 13 is a diagram that illustrates result tracking for a time period that does not meet a time-based goal and can be displayed as part of the user interface of FIG. 10 according to an embodiment;

FIG. 14 is a diagram that illustrates result tracking for a not yet completed time period and can be displayed as part of the user interface of FIG. 10 according to an embodiment;

FIG. 15 is a schematic view that illustrates a user interface that can depict a time history of medical data on a mobile device of FIG. 1 according to an embodiment;

FIG. 16 is a schematic view that illustrates a user interface that can depict a time-in-range on the mobile device of FIG. 1 according to an embodiment;

FIG. 17 is a schematic view that illustrates a user interface that can depict time-based goals on the mobile device of FIG. 1 according to an embodiment;

FIG. 18 is a schematic view that illustrates a user interface that can depict goal progress on the mobile device of FIG. 1 according to an embodiment;

FIG. 19 is a block diagram that illustrates a portion of a medical condition management system that can be part of the system of FIG. 1 according to an embodiment;

FIG. 20 is a block diagram that illustrates a configuration system that can be part of the system of FIG. 1 according to an embodiment;

FIG. 21 is a diagram that illustrates time sequencing for data management according to an embodiment;

FIG. 22 is a flow diagram that illustrates a method of data management for medical condition control that can be performed by the system of FIG. 1 according to an embodiment;

FIG. 23 is a flow diagram that illustrates another method of data management for medical condition control that can be performed by the system of FIG. 1 according to an embodiment;

FIG. 24 is a flow diagram that illustrates another method of data management for medical condition control that can be performed by the system of FIG. 1 according to an embodiment; and

FIG. 25 is a flow diagram that illustrates a method of an engagement process for medical condition control that can be performed by the system of FIG. 1 according to an embodiment.

### DETAILED DESCRIPTION

Embodiments described herein are directed to data management and interfacing for medical device usage. Embodiments may include system partitioning to reduce medical device complexity, retain the resulting data in a separate storage system for enhanced security, and provide user-facing configurability through a computer system used for engagement operations. Network traffic between a first computer system used for interfacing with medical device data and a second computer system used for user interfacing with training and engagement features can be reduced by using metrics to summarize key data values of a first time period used for data collection that may differ from a second time period used to track the metrics. Multiple data collection interfaces can be used to acquire and merge medical data associated with a medical condition, such as sensor data, pump data, and user-recorded log data that may include behavior or activity data. Further, various data entry interfaces can be supported, such as wireless data transfer, universal serial bus (USB) dongle data transfer, and direct data entry and adjustment by users and/or administrators.

Sensor data from a medical device can be collected on multiple time scales and/or include gaps in time such that values are not equally distributed in time. For instance, data collection can be muted during certain user conditions, for example, during device servicing, during data transfer operations, when disconnected or depowered, and other such instances. Embodiments may adjust for data gaps, for example, by applying time rescaling, data smoothing, data interpolation, and other such techniques. Further, as data values are compared for multiple users, the time gaps in the data can occur at different intervals. By creating one or more common metrics that summarize data for each user and potentially include different numbers of source data values, the resulting metrics can be transmitted for comparison with one or more goals at a second computer system that may not have direct access to the raw underlying data.

For example, medical devices, sensor systems, pumping systems, monitoring systems, and the like may include one or more communication interfaces to transfer data between system components. Some components with multiple communication interfaces can serve as communication conduits, for instance, to upload sensor data through and transmit over a network for storage and analysis. In some embodiments, a mobile device, tablet computer, laptop computer, wearable computing device, or other type of computing device can interface with a plurality of system components. For instance, a computing device supporting both Bluetooth and Wi-Fi may serve as a conduit to transfer data from a medical device (e.g., via Bluetooth) to a first computer system (e.g., via a Wi-Fi connection to an Internet-linked router or switch) for data storage and analytics, and the computing device may also provide an application or web-based interface to access the resulting data on a second computer system (e.g., via a Wi-Fi connection to an Internet-linked router or switch), where the resulting data includes one or more metrics summarized by the first computer system and transferred to the second computer system.

Medical devices may interface with a number of sensing and control systems. Medical device effectiveness for users can vary depending upon user behavior. Embodiments can provide users with time-based feedback relative to one or more goals and also provide engagement opportunities towards meeting future goals that may improve medical condition control, such as diabetes. The system can also educate, motivate, and inspire users through a tracking dashboard, a reward/prize system, videos, stories, quizzes, tutorials, recipes, and links to other related websites. In some embodiments, the engagement opportunities can be in the form of nominal rewards, for example, receiving points, coupons, credits, or the like, for achieving better diabetes control and/or improving control of other medical conditions. Embodiments can support the acquisition of health data associated with sensors as well as preventive health data acquired through health care system exams at clinics, hospitals, etc., and at home through do-it-yourself/self-care devices.

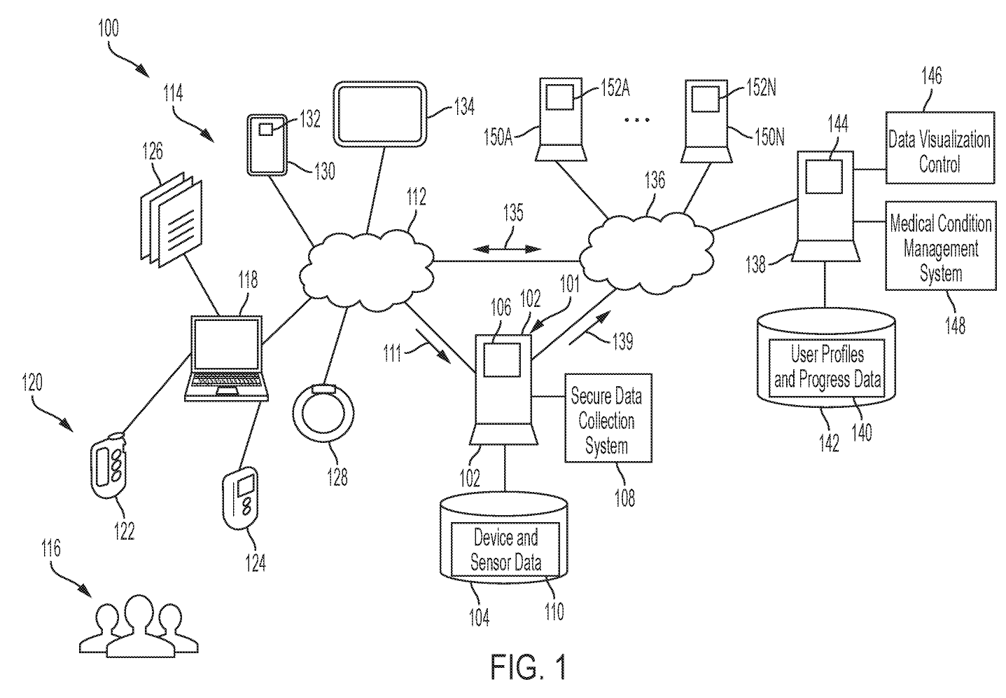

Turning now to the drawings, as shown in FIG. 1, a system 100 is illustrated, in accordance with an embodiment of the present disclosure. The system 100, or a portion thereof, can also be referred to as an engagement system 100. The system 100 can include multiple computer systems operable to transmit data, provide user interfaces, and store data at varying levels of granularity. In the example of FIG. 1, a first computer system 102 can be communicatively coupled to a data storage system 104, where the first computer system 102 includes a processing system 106 configured to perform a plurality of operations. As one example, the first computer system 102 can implement a secure data collection system 108 with respect to a plurality of device and sensor data 110 stored in the data storage system 104. The device and sensor data 110 can be uploaded 111 through a network 112 from a plurality of data sources 114 associated with a plurality of users 116 (also referred to as a community of users 116). The data sources 114 may directly interface with the network 112 or may be otherwise concentrated and merged through a computer and/or communication system operably coupled to the network 112. The first computer system 102 with data storage system 104 and processing system 106 can be collectively referred to as a server 101.

In the example of FIG. 1, a data collection computer 118 may interface with various medical devices 120, such as a pumping system 122 and a sensor system 124, where the medical devices 120 are examples of data sources 114. The pumping system 122 can be, for example, an insulin pump, a continuous glucose monitor (CGM), and/or other automated medication dosage control system. The sensor system 124 can be, for example, a blood glucose meter, an A1C measuring device, or other system that monitors a medical condition without actively controlling the delivery of medicine directly. The pumping system 122 and sensor system 124 can be part of a system that includes a glucose sensor in communication with an insulin infusion device. The data collection computer 118 may also accept input from other data sources 126, such as medical logs, reports, journals, and/or various files. The other data sources 126 may be populated by a medical professional or a patient through various testing and tracking techniques. The data collection computer 118 can be any type of computer, such as a desktop computer, laptop computer, tablet computer, mobile device, wearable computer, and the like.

The data sources 114 can also include one or more fitness sensors 128 operable to collect a plurality of fitness data, such as personal activity (e.g., steps), heart rate, blood pressure, oxygen level, and other such observable values. The data sources 114 may also include a mobile device 130 that incorporates one or more fitness sensors 132. In the example of FIG. 1, the fitness sensors 128 may be active over longer periods of time to track fitness data continuously, while the fitness sensors 132 may be accessed periodically for spot-checking of fitness data.

The data collection computer 118, mobile device 130, and/or other computing devices 134 can also communicate 135 across a network 136 to a second computer system 138, where the network 136 may be operably coupled to or combined with network 112. Rather than providing detailed medical data directly from the data sources 114 to the second computer system 138, the secure data collection system 108 of the first computer system 102 can condition and filter the medical data in summarized values. The first computer system 102 can transfer 139 the summarized values through network 136 for tracking as user profiles and progress data 140 in a data storage system 142 operably coupled to the second computer system 138. The second computer system 138 includes a processing system 144 configured to perform a plurality of operations. As one example, the second computer system 138 can implement a data visualization control 146 and a medical condition management system 148. The data visualization control 146 can generate a plurality of user interfaces to be interactively displayed on one or more user devices, such as the data collection computer 118, mobile device 130, and/or other computing devices 134. The other computing devices 134 can include any type of computing system, such as a device in a car, in a refrigerator, a phone, in a watch, in a hearing aid, in spectacles or contact lens, etc. in a visual or voice activated display that communicates data. The medical condition management system 148 can implement a patient engagement program pertinent for patients with a chronic medical, such as diabetes. One use of the medical condition management system 148 is to engage patients (e.g., users 116) in behavior changes. Behavior change may be achieved through motivational engagement in various activities (e.g., achieving health metrics goals, health management tips, education on proper use of medical devices 120, coaching, etc.) with an objective of improving health outcomes. Activities can include, for example, preventive health activities and/or self-care activities that are directly or indirectly related to an ultimate health outcome of a primary condition (e.g., more time in-range among those with diabetes). When a user engages and completes activities (e.g., by changing behavior) and/or achieves certain thresholds, the user can accrue valuable points. The points can be redeemed for education, pertinent items and/or experiences, for example.

The second computer system 138 can also interface with a plurality of linked computer systems 150A-150N, for instance, through network 136. The linked computer systems 150A-150N can include corresponding processing systems 152A-152N. The linked computer systems 150A-150N can provide access to one or more linked assets and/or provide reward tracking and redemption services. Linked assets may include materials to support education of the users 116 and interaction between the users 116, such as social media content relevant to one or more shared medical conditions of the users 116. Engagement opportunity reward tracking and redemption can enable users 116 to redeem points accrued through the medical condition management system 148. For instance, points may be converted to credit for online purchases, discounts, tickets for an experience, and other such participation incentives of nominal value.

In embodiments, the medical condition management system 148 can apply a motivational model or other such model that focuses on health outcomes, not just health activities of the users 116. For instance, the medical condition management system 148 can apply various gamification activities and challenges, such as achievement, maintenance or improvement of health-related outcomes (e.g., A1C, blood sugar time-in-range, etc.). Gaming mechanics can include data visualization of time-in-range data, challenges related to a health-related outcome, a tiering system to track performance and receive encouragement to “level-up” to the next highest tier, surveys and quizzes with aggregated insights, and peer-to-peer interactions amongst a community to compare progress against health-related challenges, and/or user-generated content on time-in-range success stories that may serve as inspiration for other members. The data visualization control 146 is operable to display health outcome achievements to individual users 116 and/or across the community of users 116. This provides a substantially instantaneous feedback loop system to further engage the users 116 to change behavior, complete more activities and strive for continued better health outcomes, thus forming/increasing healthy habits. Further, challenges can be designed to encourage patients to report, complete and achieve activities and outcomes beyond a primary condition (e.g., going beyond diabetes to decrease obesity, etc.).

Various health-related outcomes can be tracked by the medical condition management system 148 based on data collected from the data sources 114. In the context of measuring diabetes-related health outcomes, metrics can include percentage time in range (sensor glucose), percentage time in low range, percentage time in high range, low glucose events, high glucose events, A1C, mean average glucose, glucose management indicator, glycemic variability, and the like. Although described primarily with reference to diabetes, it will be understood that other medical conditions can also or alternatively be monitored with corresponding health condition modifications sought using the system 100. Further, although described and depicted in FIG. 1, as separate computer systems, in some embodiments, the secure data collection system 108, the data visualization control 146, and/or the medical condition management system 148 can be further subdivided or combined. For example, a localized embodiment may execute on the data collection computer 118, mobile device 130, and/or other computing devices 134 for a subset of the users 116, where a single device or system may support a single user 116 or a group of users 116. Further, the secure data collection system 108, the data visualization control 146, and/or the medical condition management system 148 may have application programming interfaces to support modular incorporation with other systems, for instance, to further customize data visualization, goals, challenges, engagement opportunities, and the like. Any type of user interfaces can be supported, including but not limited to, graphical interfaces, audio interfaces, voice-based interfaces, text-based interfaces, virtual or augmented reality-based interfaces, and other such interfaces known in the art. Further, one or more of the computer systems depicted in FIG. 1 can be implemented in one or more cloud computing resources, where the cloud computing resources can be provisioned from shared pools of configurable resources with services provided as needed over one or more computer networks.

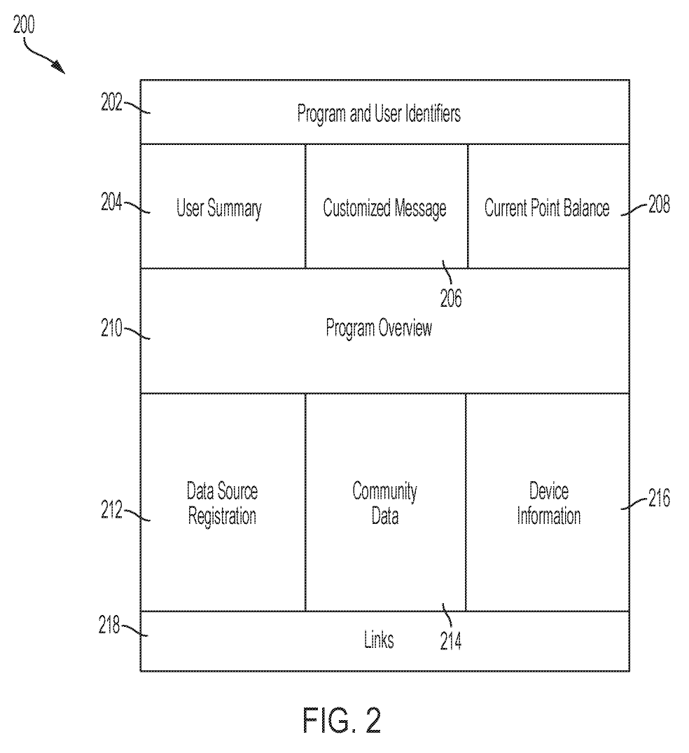

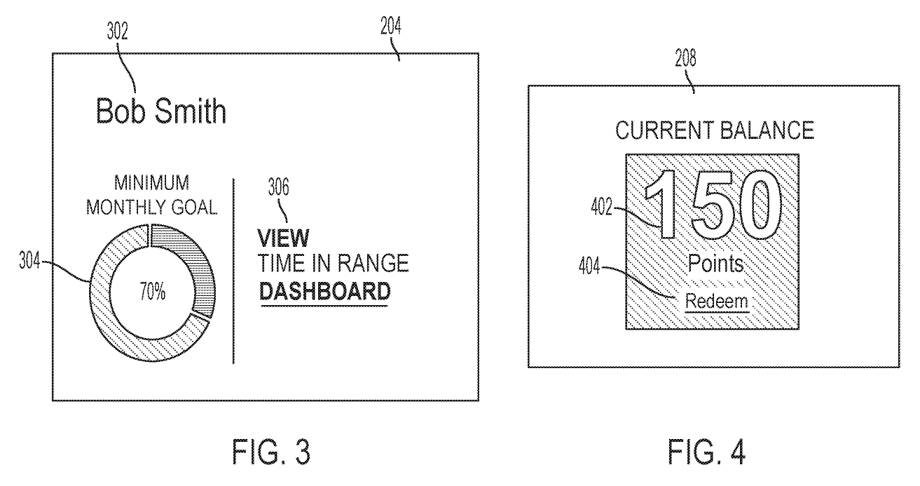

FIG. 2 is a block diagram that illustrates a user interface 200 according to an embodiment. The user interface 200 can be created by the data visualization control 146 and displayed by one or more of the data collection computer 118, mobile device 130, and/or other computing devices 134 of FIG. 1, for instance, through a web-enabled interface or app. In the example of FIG. 2, the user interface 200 includes program and user identifiers 202 to indicate which user is actively logged in and to whom the associated user data pertains. A user summary 204 can provide further user information, such as the example of FIG. 3, which includes a user name 302, a goal 304, and one or more links 306 (e.g., to view a time-in-range dashboard). With continued reference to FIG. 2, the user interface 200 can include a customized message 206, such as a tip of the day, and a current point balance 208 of the user. An example graphical depiction of the current point balance 208 is depicted in FIG. 4, where a current number of points 402 is provided in combination with a redemption link 404. The redemption link 404 may trigger a transition to a redemption server of the linked computer systems 150A-150N of FIG. 1, where redemption and point management operations can be performed through a different interface or as an embedded component of the current interface. User credentials and point data can be handled similar to other types of sensitive/financial data. Redemption activities result in a corresponding reduction to the current number of points 402.

Further regarding FIG. 2, the user interface 200 can also include a program overview 210 that describes how to interface with elements of the medical condition management system 148 of FIG. 1. The program overview 210 may include a video, slides, audio description, questions and answers, and other such instructive materials that may result in earning points upon completion as a training/education activity. The user interface 200 can also include a data source registration 212, community data 214, device information 216, and links 218. Interactions with the data source registration 212, community data 214, and device information 216 may also result in earning points upon completion. For example, the data source registration 212 can include configuration instructions and a link to configure the data collection computer 118 and/or the first computer system 102 to acquire the device and sensor data 110, as well as scheduling the transfer 139 from the first computer system 102 to the second computer system 138 of FIG. 1. The community data 214 can include video, audio, text, and/or other related content shared by one or more of the users 116, for example, that may relate to using one of the medical devices 120 of FIG. 1. The device information 216 can include tips or suggestions related to one of the medical devices 120 of FIG. 1 or other related information. The links 218 may include hyperlinks to other content and web pages, including, for instance, hyperlinks to social media, frequently asked questions, contact information, and other such information. Although the example of FIG. 2 depicts one configuration of the user interface 200, it will be understood that many variations are possible, including many types of additional supporting resources (not depicted).

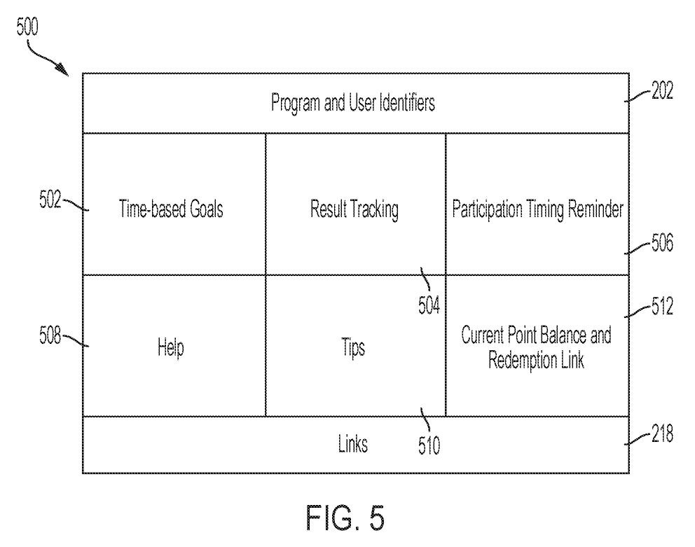

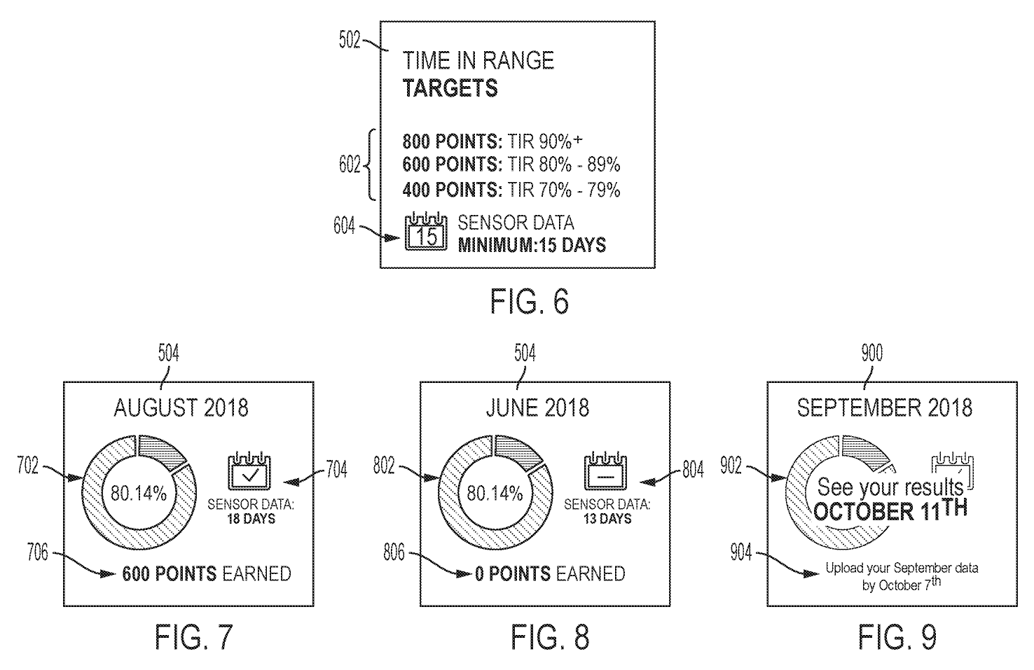

FIG. 5 is a block diagram that illustrates a user interface 500 according to an embodiment. The user interface 500 is an example of a time-in-range dashboard that may be reached through the one or more links 306 of FIG. 3. Similar to FIG. 2, the user interface 500 can include the program and user identifiers 202 and links 218. The user interface 500 can also include time-based goal tiers/levels 502, result tracking 504, participation timing reminder 506, help 508, tips 510, and current point balance 512—similar to FIG. 4. The time-based goal tiers 502 can include goal tier definitions and engagement reward points available 602 and a minimum sensor data qualification requirement 604 as depicted, for example, in FIG. 6, where three goal and point tiers are defined along with a minimum sensor data qualification. The goal tiers and engagement reward points available 502 can include, for example, differing values of engagement rewards issued for meeting one of the goal levels described. For instance, an engagement reward of 800 points is available for achieving a health outcome of staying within a lower limit and upper limit defined as being “in-range” for at least 90% of each month the user participates, 600 points for staying in range 80-89% of the month, and 400 points for staying in range 70-79% of the month. The minimum sensor data qualification requirement 604 specifies how many days the user must have worn a glucose sensor to qualify for any engagement reward points. The help 508 may include links to information on how to use the time in range dashboard of user interface 500 and how to earn points through engagement with the medical condition management system 148. The tips 510 may include links on how to use the medical devices 120 of FIG. 1, how to stay “in-range”, and other such information.

FIGS. 7 and 8 depict examples of the result tracking time period summary 504 of FIG. 5 respectively. In FIG. 7, the result tracking time period summary 504 is depicted for the month of August, including resulting values for a time-in-range metric 702, a sensor data metric 704, and an engagement points reward earned 706. In the example of FIG. 7, the time-in-range metric 702 falls within the 80-89% value of the middle tier time-based goal 602, which results in the user earning a nominal 600 points engagement reward as indicated in the engagement reward earned 706. The sensor data metric 704 confirms for the user whether or not the minimum sensor data qualification requirement 604 was met for the month. In contrast, FIG. 8 depicts an example where the time-based goal 602 was met, but the minimum sensor data qualification requirement 604 was not. As can be seen in FIG. 8, for the month of June, the user had a time-in-range metric 802 that was within the middle tier range of 80-89% of the time-based goal 602, but the user did not supply enough sensor data to meet the 15 day minimum data requirement 604, which results in the user earning no engagement reward points as indicated in the reward earned 806. FIG. 9 depicts an example of a summary display 900 where results are not yet available as indicated by a status message 902 and an action prompt 904 to initiate a data upload by a specified date. The action prompt 904 notifies the user of actions to take, for instance, with respect to the data collection computer 118 and the first computer system 102 such that data needed at the second computer system 138 is available for analysis at a desired time. The summary display 900 can be displayed as part of a user interface, such as the user interface 500 of FIG. 5, for example.

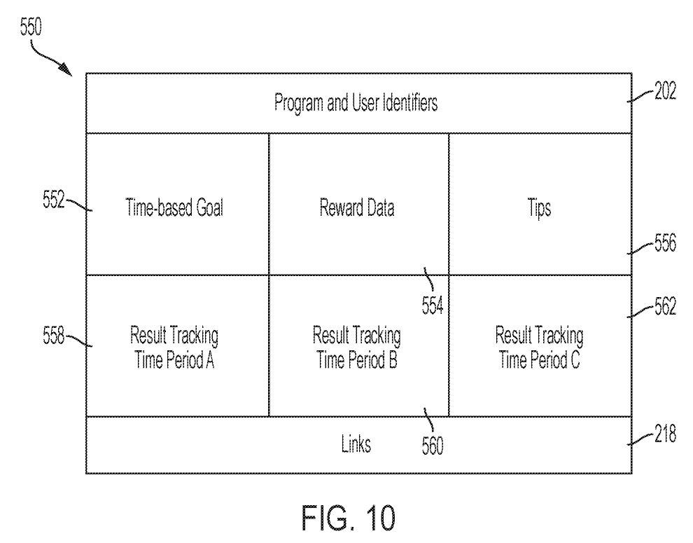

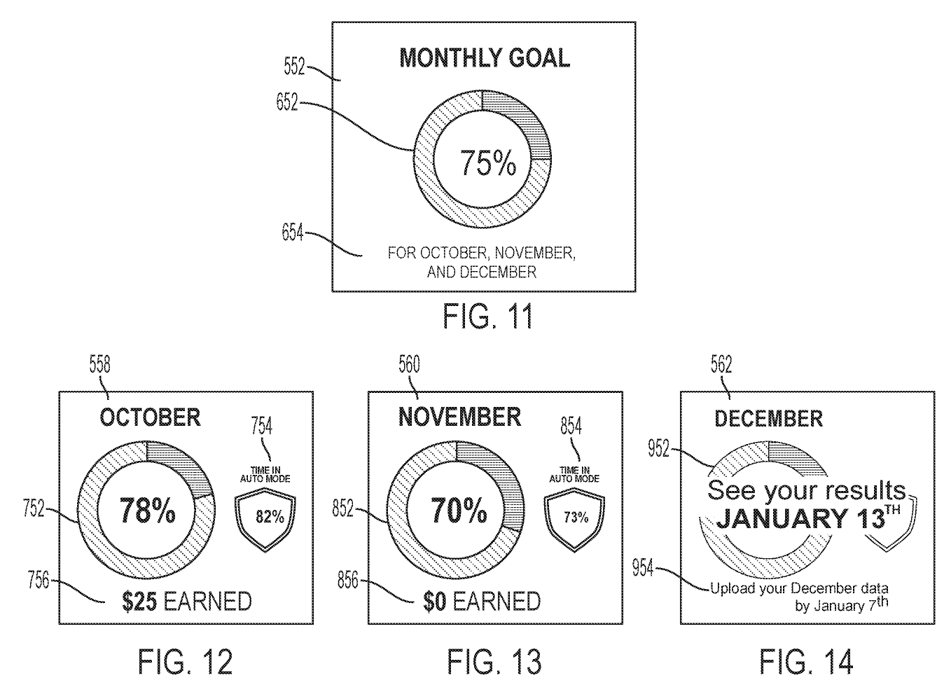

FIG. 10 is a block diagram that illustrates a user interface 550 according to an embodiment. The user interface 550 is an example of a time-in-range dashboard that may be reached through the one or more links 306 of FIG. 3. Similar to FIG. 5, the user interface 550 can include the program and user identifiers 202 and links 218. The user interface 550 can also include a time-based goal 552, reward data 554, tips 556, and multiple result tracking time period summaries 558, 560, 562. The time-based goal 552 can include a goal graphical depiction 652 and a goal time range 654 as depicted, for example, in FIG. 11, where a monthly goal of 75% time-in-range is established for the months of October, November, and December. The reward data 554 can include, for example, a graphical depiction of the value of a reward issued for meeting the goal described in the time-based goal 552. For instance, a twenty-five dollar reward applicable to a retailer desired by the user may be available for achieving a health outcome of staying within a lower limit and upper limit defined as being “in-range” for at least 75% of each month of the goal time range 654. The tips 556 may include links on how to use the medical condition management system 148, how to use the medical devices 120 of FIG. 1, how to stay “in-range”, and other such information.

FIGS. 12-14 depict examples of the result tracking time period summaries 558, 560, 562 of FIG. 10 respectively. In FIG. 12, the result tracking time period summary 558 is depicted for the month of October, including resulting values for a time-in-range metric 752, a time-in-auto-mode metric 754, and a reward earned 756. In the example of FIG. 12, the time-in-range metric 752 is greater than the 75% value of the time-based goal 552, which results in the user earning a $25 reward as indicated in the reward earned 756. The time-in-auto-mode metric 754 may be instructive to the user to understand how the time-based goal 552 was met even if the time-in-auto-mode metric 754 is not explicitly part of the time-based goal 552. In contrast, FIG. 13 depicts an example where the time-based goal 552 was not met. As can be seen in FIG. 13, for the month of November, the user had a time-in-range metric 852 that was less than the 75% value of the time-based goal 552, which results in the user earning no reward as indicated in the reward earned 856. It is noted that a time-in-auto-mode metric 854 had a value of 73%, which is below the time-in-auto-mode metric 754 of 82%. FIG. 14 depicts an example where results are not yet available as indicated by a status message 952 and an action prompt 954 to initiate a data upload by a specified date. The action prompt 954 notifies the user of actions to take, for instance, with respect to the data collection computer 118 and the first computer system 102 such that data needed at the second computer system 138 is available for analysis at a desired time.

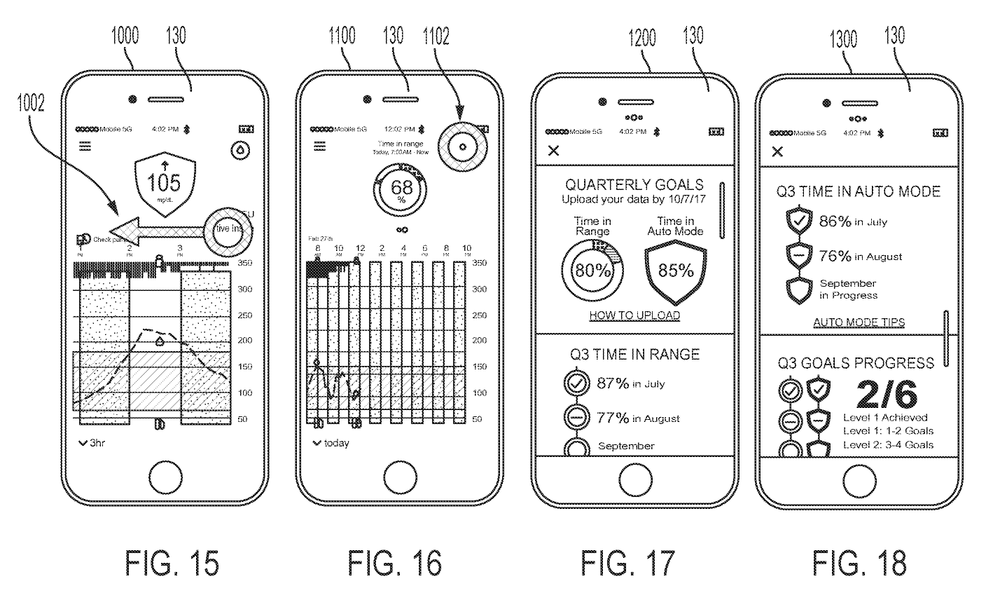

FIGS. 15-18 illustrate user interfaces on the mobile device 130 of FIG. 1 according to embodiments. A user interface 1000 on the mobile device 130 can depict a time history of medical data, for example, as received from one or more of the medical devices 120 of FIG. 1. Touching a location or making a swiping gesture 1002 on the user interface 1000 can result in transitioning to another display output, such as user interface 1100 of FIG. 16. In the example of FIG. 16, a historic plot of a time-in-range is depicted. The time-in-range as depicted in the user interface 1100 may be computed using a user-selected time base or a recent history range, such as a daily value. An icon or other indicator 1102 may be selectable on the user interface 1100 to trigger access to the data visualization control 146 to further interface with the medical condition management system 148 on the second computer system 138, resulting in a transition to user interface 1200 of FIG. 17. As can be seen in the transition between the user interfaces 1100 and 1200, the difference in time scale shows that from previously summarized and uploaded monthly data, the user achieved 87% time-in-range in July which met the monthly goal of 80% and 77% in August which did not meet the monthly goal. The current daily value of 68% time-in-range depicted in the user interface 1100 may not yet be available to the second computer system 138 until the data is uploaded and further processing and summarized through the first computer system 102 of FIG. 1. FIG. 18 further depicts a user interface 1300 that illustrates a time-in-auto-mode value of 86% in July that met the monthly goal of 85% (as depicted in user interface 1200) and a time-in-auto-mode value of 76% for August that did not meet the 85% goal. Upon exiting the user interface 1300, the mobile device 130 may revert to the user interface 1100 from which the user interface 1200 was launched.

Various program elements, such as the time-in-range goal activity, program assets and content, and the like, can be displayed within other programs and applications through a modular approach. These modules can take individual program elements or combinations thereof and display them within other programs and applications to simplify or enhance user experience. For instance, a display of program elements in other programs and applications can utilize native functionality of the other application, for example, user interface 1000 displaying medical data from sensor system 124, such as immediate access to time-in-range data that can feed into the user interface 500, time-based goal tiers/levels 502, and result tracking 504. In this example, the user can view results against goals without having to exit the program or application in which the module is placed. Similarly, relevant program assets and content can be displayed through a module, allowing a user to view the assets and content without having to exit the program or application in which the module is placed. A module can also inform functionality of the program or application in which it is placed. For example, in an application that provides messages or notifications to a user, the module can trigger a message to the user that informs the user of a result or goal achieved, a motivational message to improve a result, a command to complete an action, a notice of a goal that is not achieved, an asset or piece of content, or an insight based on myriad data sources available within the module and the application.

Behavioral model-based triggers can enable proactive interactions with users. For example, based on an analysis of data, statistical algorithms and machine learning, embodiments may identify data patterns, insights, and status of where a patient stands in terms of the health outcome challenge, a number of points, standing in a “leader board”, etc. and can trigger a message (e.g., voice, text, letter, call from coaches, notification in a website or app, etc.) to alert, educate, and/or challenge the user with new data information, knowledge, learning, insights, and wisdom. New data information, such as a summary of personal health care data (e.g., time in-range, time below range, mean glucose, weight, hypertension, etc.), a summary of the number of points, a reminder to redeem the points in a marketplace, can be provided. Trigger conditions can be checked multiple times during the day, during the week, during the month, etc. Notifications may take the form of a picture in time, of a picture over time, of a comparative picture over best or worst points in time, etc. Conversely, the patient may also rate or rank trigger and motivational messages which can be used to further enhance underlying models and processes (e.g., a patient feedback loop system of satisfaction and usefulness).

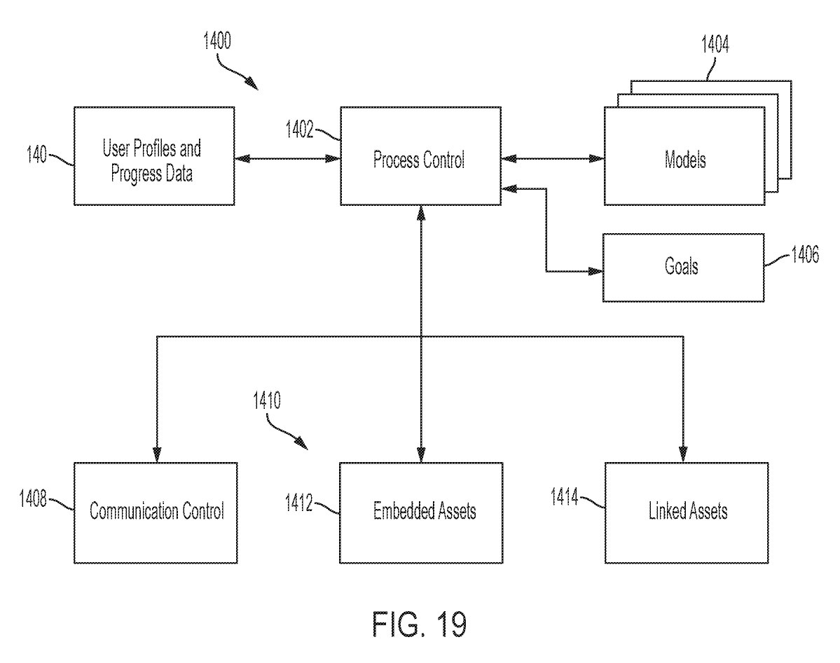

FIG. 19 is a block diagram 1400 that illustrates a portion of the medical condition management system 148 of FIG. 1 according to an embodiment. A process control 1402 can access the user profiles and progress data 140 from the data storage system 142 and access one or more models 1404 to determine one or more goals 1406 to modify a health-related outcome of the community of users 116 over a period of time. Examples of the models 1404 include behavioral models that can send a motivational message or trigger an ability to act out/complete the behavior in a finite period of time. The models 1404 and corresponding goals 1406 drive a desired behavior related to better health compliance and usage around a medical condition or a medical management device (e.g., medical devices 120 of FIG. 1), such as a CGM or a medication delivery system, such as an insulin pump or insulin pen. For example, a behavioral model can be operationalized for a patient-user as follows: the process control 1402 can access a communication control 1408 to send a message through a communication channel, such as an email or a text message alerting or reminding the user to complete an activity (i.e., related to a positive health outcome). An email or text channel may contain a “motivational or inspirational” set of messages or may contain an invitation to visit a website of the medical condition management system 148 or an app or another secondary messaging channel. The content/topic of a motivational message can be related to the management of the medical condition (e.g., about food, exercise, stress, sleep, etc.) or the health management device (e.g., tips on how to best operate the device, such as best times to give an insulin bolus, how to adjust basal rates, etc.).

The content/tone of the motivational message may create awareness to basic motivations such as fear (e.g., avoidance of complication in diabetes), hope (e.g., surviving to the age that can enjoy grandchildren), pain (e.g., not feeling well), gratification (e.g., immediate reward in the form of prizes), and the like, which can be personalized to declared or inferred preferences of each of the users 116 of FIG. 1. The message content may also contain motivational reminders pertaining to gamification challenges, such as patients' standing against a leaderboard, or number of accumulated points or how close to achieving a certain goal and the further accumulation of points. The message may also contain a “call to action” that further encourages completion of the behavior; e.g., “act now”, “check your time-in-range now”, etc. The combination of a “trigger/reminder” plus “motivational message” may catalyze the user into an action/behavior that is relatively simple to complete, i.e., the user should have the ability to enact the desired behavior, preferably immediately. For example: an email message may contain, “Read autumn soup recipe and earn points. Helps with sugar variability. It's simple. Make it tonight”.

The process control 1402 can provide access to a plurality of assets 1410 through a plurality of communication channels to a community of users 116 associated with a medical condition. The assets 1410 can include one or more embedded assets 1412 locally accessible by the second computer system 138 and one or more linked assets 1414 accessible through network 136 operably coupled to the second computer system 138. The assets 1410 can support the completion of various activities that may result in accumulation of points and impact the health outcomes of the users 116.

The process control 1402 can engage users 116 through a variety of channels, including social, digital, and other mass media channels and multiple display devices, for instance, through the communication control 1408. The communication control 1408 can send notifications in social media, phone calls, emails, text messages, etc. Users 116 can access the medical condition management system 148 through a website, apps via a computer, a smartphone, a wearable device, and other readily accessible devices that can visually or via audio present activities or status updates related to the health outcome or the points system or the redemption of points. The medical condition management system 148 may also be accessible as an integral part of therapy devices used to manage a medical condition (e.g., an insulin pump or remote controller that controls an insulin pump). Thus, the users 116 can receive alerts, notifications, challenges, updates, through a variety of communication channels.

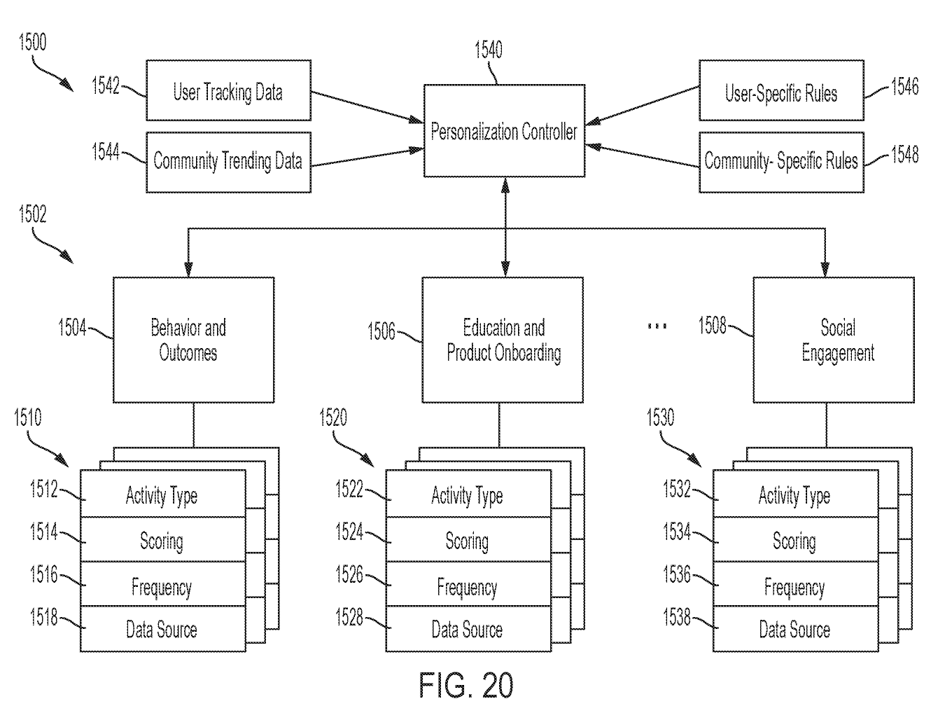

A user can complete many types of activities to earn points. The activities can be related to a health condition or to a management device (or treatment therapy). The variety and ability to select activities can increase motivation and engagement. The activities may improve the wellness and/or health of the user. The activities may take the form of articles, videos, surveys, quizzes, testimonials, and other such materials accessible through the assets 1410. Detail configuration of the activities and sequencing of the goals 1406 in support of the models 1404 can be managed through a configuration system 1500 as depicted in FIG. 20.

In the example of FIG. 20, a plurality of activities 1502 can be partitioned or subdivided into categories, such as behavior and outcomes 1504, education and product onboarding 1506, and social engagement 1508. Behavior and outcome activity options 1510 of the behavior and outcomes 1504 may be configured to define an activity type 1512, such as registration and data uploading, scoring 1514 that defines available points for the activity, a frequency 1516 that defines how often the activity is available for completion, and a data source 1518 that defines whether embedded assets 1412 or linked assets 1414 of FIG. 19 are accessed for the activity. Education and product onboarding options 1520 of education and product onboarding 1506 may be configured to define an activity type 1522, such as course completion, reading an article, watching a video, taking a quiz, and/or activities that lead to more expedient and successful utilization of targeted products. The education and product onboarding options 1520 can also include scoring 1524 that defines available points for the activity, a frequency 1526 that defines how often the activity is available for completion, and a data source 1528 that defines whether embedded assets 1412 or linked assets 1414 of FIG. 19 are accessed for the activity. Similarly, engagement options 1530 for social engagement 1508 may be configured to define an activity type 1532, such as reading/watching a personal story, writing a personal story, taking a survey, sharing tips, engaging a social media channel, and other such activities. The engagement options 1530 can also include scoring 1534 that defines available points for the activity, a frequency 1536 that defines how often the activity is available for completion, and a data source 1538 that defines whether embedded assets 1412 or linked assets 1414 of FIG. 19 are accessed for the activity.

Collectively, the content of the activities 1502 may be educational, fun, news, informative, etc. The activities 1502 may engage an individual user or a community of users 116 (e.g., more than one user). The activities 1502 can be related to driving a desired behavior which helps improve wellness and/or a health outcome. An example of behavioral activities (e.g., in behavior and outcome activity options 1510) that result in better health outcomes is described for an insulin delivery system as follows. Users 116 can be challenged on a daily, weekly, monthly quarterly, six months or annual basis to achieve certain health outcomes, such as “percentage time-in-range” (>75%) or A1C (<7), tracking time in auto-mode on pumping system 122 of FIG. 1 or tracking a food log or journal to help with blood glucose control. This may be an important element of motivation because it is both extrinsic (e.g., accumulation of points) and intrinsic (e.g., sense of accomplishment) to each user.

In the case of diabetes, health guidelines related to type-one and type-two diabetes can include a combination of activities related to food, diet, exercise, portion control, and medication adherence. For instance, eating smaller portions and healthy foods can help prevent or delay diabetes diagnoses. Physical activity can help control blood glucose levels, weight, and blood pressure, as well as raise “good” cholesterol and lower “bad” cholesterol. Losing just a small amount of weight (e.g., between 5 and 7 percent of total body weight) can prevent or delay type-two diabetes for those who are at high risk for the disease. Thus, activities 1502 can target these and other goals.

Health outcome challenges can take different forms of motivation and have different values of points, as defined, for example, in scoring 1514, 1524, 1534. Further, the user may be challenged to achieve or to maintain certain health outcome thresholds. In additional embodiments, the thresholds may change (including increasing and/or decreasing as appropriate) to make the challenge more difficult than the prior challenge to further improve outcomes and/or motivate the user 116. Challenges may also take the form of competitions or team challenges, depending on the user preference. These can involve competing with fellow members on highest time in range, highest improvement in time in range, and/or competing on number of points earned in a given month. The challenges may also vary depending on varying time frames. The users 116 may take on multiple challenges. In some instances, the users 116 may change their challenge. Challenges may be offered continuously or on a temporary/promotional basis. Outcomes can be expanded beyond a primary condition to other related disorders or conditions. With respect to diabetes, increased time in-range and better health outcomes can be encouraged through activities 1502, such as visiting doctor frequently, engaging with health community workers, frequently measuring and monitoring diabetes adverse implications, such as foot checks, eye exams to measure retinopathy, and other preventive health activities such as flu shots and immunizations. As a further example, a patient with diabetes may be educated, motivated and rewarded for reporting and achieving better time in range or lower A1C or less time in high range, etc. (a primary condition) and additionally may also be challenged and rewarded for lower weight, lower hypertension, cholesterol level, sleep, kidney disease, etc. (comorbidities).

Coaching activities can also be supported to interface users with human or artificial intelligence-based coaches. Health and therapy coaches may intervene at appropriate time points to challenge a patient using an established and recognized treatment protocol. Coaches may modify and adjust health outcome challenges based on the needs of the patient and within certain parameters. The coaches may look at and interpret individual patient data and proactively or reactively intervene and support the patient. Coaches may analyze population data and identify and intervene with patients at greater risk or simply reward those that are doing their best or making the highest effort.

An absolute health outcome goal based on populations distributions can include where users 116 may be challenged to achieve one of the goals 1406 which has been predetermined, for instance, based on probabilistic population distributions of patient health outcomes. For example, if the average type 1 diabetes population has a time-in-range of 60%, then a time-in-range challenge may be set up half a standard deviation higher or X % higher so that the majority of the population will need to engage in a new behavior to meet the threshold. Health outcome goals may also be set up based on public health policy guidelines set up by credible organizations. American Diabetes Association (ADA) recommendations are an example, in diabetes, of a source of data for constructing the goals 1406.

In embodiments, relational goals can be established by a personalization controller 1540 accessing user tracking data 1542, community trending data 1544, user-specific rules 1546, and/or community-specific rules 1548 to perform personalization for a user relative to individual or community health outcomes. The user tracking data 1542 can establish a history of meeting goals, rates of change, missed goals, and other such information. The community trending data 1544 can include averages from the community of users 116 and may identify patterns of general improvement or regression. User-specific rules 1546 can define user-specific challenges, while community-specific rules 1548 can define challenges for the community of users 116.

For a relative personal health outcome goal, users 116 may be challenged to achieve a predetermined percentage or absolute value improvement over a previous health outcome before using the system 100 of FIG. 1 or based on a preset initial baseline. For example, if a type 1 diabetes patient has a time-in-range of 60% before using the system 100, then the patient may be challenged to achieve a 10% improvement (i.e., 66% time-in-range) or to achieve a ten-point improvement (i.e., 70% time-in-range) over a certain time period (a month, 3 months, etc.).

For a relative to population health outcome goal, users 116 may be challenged to achieve a certain health outcome based on deciles distribution of similar populations. For example, a type 1 diabetes patient may be challenged to achieve (or maintain) the top 90% decile for time-in-range. “Level up” challenges can be tuned to specific users 116, where some users may be challenged to improve by 5%-25%, for example, depending on various individual achievements attained.

For a personalized health outcome goal, users 116 may set their own challenges. The value of a challenge can have a difficulty index and an accordingly proportional value. For example, a type 1 patient may set a goal of achieving a 10% improvement of time-in-range over 12 months. Another patient may start with 30% time-in-range and set a challenge to achieve 90% time-in-range in three months. Another patient may set a challenge to maintain 90% time-in-range for the next 6 months. Each of these challenges has a difficulty and an associated point value.

A patient can earn points for completing activities 1502 and achieving certain health outcome thresholds. This can be important in motivation by driving primarily health outcomes, not just completion of the activities 1502. The point system can be weighted to achieving health outcomes, meaning that a patient can earn more points when the highest possible or a healthy outcome is achieved. Health outcomes may be tiered in terms of points. Activities 1502 that are wellness related or help in the management of the condition or the management of therapy may earn fewer points.

As an example, in a month, a user can earn up to a certain number of points which may have a certain nominal monetary value or some other value. A ratio of points may be constrained at least >50% towards health outcomes thresholds. In other words, users 116 can earn a majority of the points through achieving health outcomes, not by completing activities 1502. Points can also be accrued, which may have no monetary value, but have a probabilistic (game of chance) opportunity to earn a certain value.

In some embodiments, users 116 can redeem accumulated points for engagement rewards at any time, e.g., through a redemption interface. Users 116 may have the ability to select engagement rewards from multiple health-related categories, including products, services, experiences, philanthropic donations, reduction in copays and deductibles, etc.

The personalization controller 1540 can support different approaches for personalization. As one example, a user can declare a set of preferred options which may be stored in the user-specific rules 1546. The user can select preferences based on a Q&A dialogue, which may be created through a user interface. The user may also be able to customize a preferred set of activities 1502 to earn points. The user may also be able to set a health outcome challenge. The user may further be able to select the frequency of news and notifications and a level of engagement. Activity types and tone of messaging may also be selectable by the user.

Conversely, the medical condition management system 148 can be automatically personalized to the user based on segmentation and cluster analysis of populations and factors such as behavior, attitudes, motivations, demographics and psychographics, etc. The medical condition management system 148 can also be personalized based on a set of rules using artificial intelligence, such as adjusting the user-specific rules 1546. An aim may be to provide content, challenges, and the like that can further engage and motivate the user to take actions around learning and managing the condition and the therapy. Further, the medical condition management system 148 can be personalized around the areas of content, challenges, user interfaces, rewards, and other such categories.

Embodiments may administer, deliver and monitor the integration and efficacy of programs that are academically proven and recommended by recognized health care systems. Thus, processes and systems of challenging patients may also be applied to recognized care pathways as part of the medical condition management system 148. Further, the medical condition management system 148 may identify and uncover new care pathways based on statistical and usage of artificial intelligence to learn and optimize challenges, and rewards and activities that create greater patient engagement and the achievement of better health outcomes. Programing and sequence of certain activities, the setup of certain challenges and type of health outcome goals, the points and rewards system may evolve based on data algorithms can allow for segment customization or individual personalization.

The personalization controller 1540 can interface with the data visualization control 146 to impact one or more user interfaces of the medical condition management system 148 for personalization and engagement. The personalization controller 1540 can select a combination of visual and/or audio content to represent health outcomes in a manner that shows progress with respect to a challenge. Gamified environment aspects of health outcomes can include leaderboards, journey narratives, patient selection of goals, achievement of certain levels, earning of “badges”, and other interfaces that encourage continued interactions by the users 116. Embodiments can aggregate (e.g., longitudinally) multiple sources of data (e.g., sensors, doctor exams, hospital visits and claims, etc.) related to multiple associated conditions (diabetes, obesity, etc.) as well as self-reported measures (patient reported outcomes, quality of life measures, patient therapy preferences, condition and therapy attitudes and behaviors, etc.) and identify the best engagement (e.g., the best combination and sequence of activities, challenges, rewards) for each individual patient or patient segment. Optimization and machine learning can be applied over time as a patient adjusts and adapts attitudes and behaviors. Data management can simulate theoretical experiments to determine an optimization for delivering actual programs to users.

In embodiments, the medical condition management system 148 can also engage users 116 by providing content, awards, and/or challenges based on an ecosystem of partners that are integrally related to the desired health outcomes. For example, the medical condition management system 148 can gamify a time-in-range challenge by partnering with a “better sleep company” through the linked computer systems 150A-150N of FIG. 1 and create a unique challenge around time-in-range at night. This interrelatedness may further motivate users 116 to achieve better health outcomes. As users 116 accumulate points, a redemption process may take a basic approach as well as a gamified approach in which users 116 can redeem points for products, services, experiences, or donation of all/some points to a charity, for instance, through interactions with the linked computer systems 150A-150N. Users 116 may also barter points to trade certain items through the medical condition management system 148 and/or the linked computer systems 150A-150N of FIG. 1. Further, the education and product onboarding 1506 and social engagement 1508 can encourage facilitation of conversations, sharing of tips, and information within the community of users 116. Interactions between the users 116 can be constrained according to the community-specific rules 1548 and may be facilitated through the medical condition management system 148 and/or the linked computer systems 150A-150N.

As an example of results observed when implementing an embodiment of the system 100 of FIG. 1 for a group of users 116, a sample group of the users 116 was classified into groups of high engagement, medium engagement, and low engagement with respect to completion of the activities 1502. As an example, users 116 completing less than 10% of the activities 1502 may be classified as low engagement, users 116 completing 25% or more of the activities 1502 may be classified as high engagement, and remaining users 116 can be classified as moderate engagement. In examining at least one diabetes health outcome metric (e.g., time in range) of the users 116, members of all three groups showed an increase in the at least one diabetes health outcome metric as engagement continued over an extended period of time (e.g., multiple months). Thus, the system 100 can have an effect on a particular treatment for diabetes, particularly in conjunction with implementing one or more of the processes described herein.

In embodiments, the system 100 can use various integrity checks to authenticate and secure one or more sources of data. For example, block chain can be used for integrity certification to ensure that a patient has indeed completed certain activities, earned a certain amount of points and redeemed points for rewards and recognitions over time. In another example, block chain can ensure the integrity of data being shared across multiple partners that support and share patient data. For instance, the system 100 of a portion thereof may be implemented in a hospital organization, and the resulting data may be shared with a health insurance payer and transmitted to a third party to administer rewards, where block chain may ensure secure transmission of data across multiple entities. Block chain or related technologies can ensure that patients are rewarded for achieving health outcomes, following a care path plan, or adhering to therapy recommendations. Further, patients can also be rewarded for sharing data in the system 100, and block chain can facilitate and recognize such a transaction, particularly where transfers of value occur in the form of incentives and other forms of recognition. Since the system 100 can integrates multiple data sources, block chain may further facilitate stitching of varied data sources while maintaining security and data integrity.

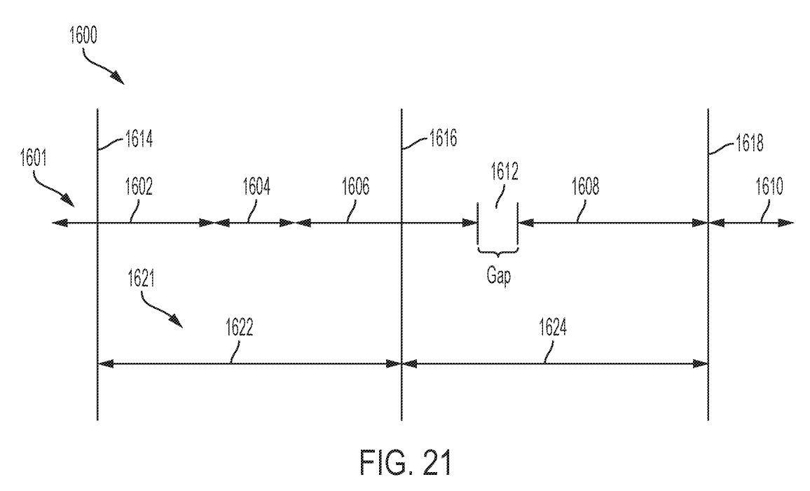

FIG. 21 illustrates time sequencing 1600 for data management according to embodiments. In managing data movement from the data sources 114 to the second computer system 138 of FIG. 1, the first computer system 102 can act as an intermediary to securely store sensitive data and limit the type of data, amount of data, and volume of data made available to the second computer system 138 and/or the linked computer systems 150A-150N. Data from the data sources 114 may arrive at the first computer system 102 in varying time increments depending upon how frequently an upload 111 is initiated from the data sources 114. For example, the device and sensor data 110 may be received from the data sources 114 in a first time sequence 1601 that includes data block 1602, 1604, 1606, 1608, and 1610 that span a period of time. Each of the data blocks 1602-1610 may have different quantities of data depending upon a level of activity with sensed data, and an upload interval. For example, data block 1602 may span a two-week period that splits between calendar months, data block 1604 may be a six-day period within a month, and data block 1606 may be a fifteen-day period that splits between two months. Further, when a user does not regularly use medical devices 120 of FIG. 1, there can be gaps in the time sequence data, such as a gap 1612 between data blocks 1606 and 1608. The gap 1612 may span for multiple days, for instance.

The example of the first time sequence 1601 illustrates variability that may exist for a single user, while multiple users 116 can have many such variations collectively. In embodiments, to support data normalization, scheduling of transfers 139, and to reduce traffic on the network 136 of FIG. 1, the first computer system 102 can establish a standardized reporting time period, such as once per month. Accordingly, time sequence 1621 includes a first data block 1622 between time intervals 1614 and 1616, and a second data block 1624 is between time intervals 1616 and 1618. The first data block 1622 summarizes data from a portion of data block 1602, all of data block 1604, and a portion of data block 1606. The second data block 1624 summarizes data from a portion of data block 1606 and a portion of data block 1608. The time intervals 1614, 1616, and 1618 can be established as common limits across the device and sensor data 110 for all of the users 116. Thus, uploads 111 of data from the data sources 114 of FIG. 1 to the first computer system 102 can be asynchronous to the transfer 139 of data to the second computer system 138. Staggered scheduling of the transfers 139 to the second computer system 138 can ease the processing burdens and storage system bottlenecks of the data storage system 142, for example.

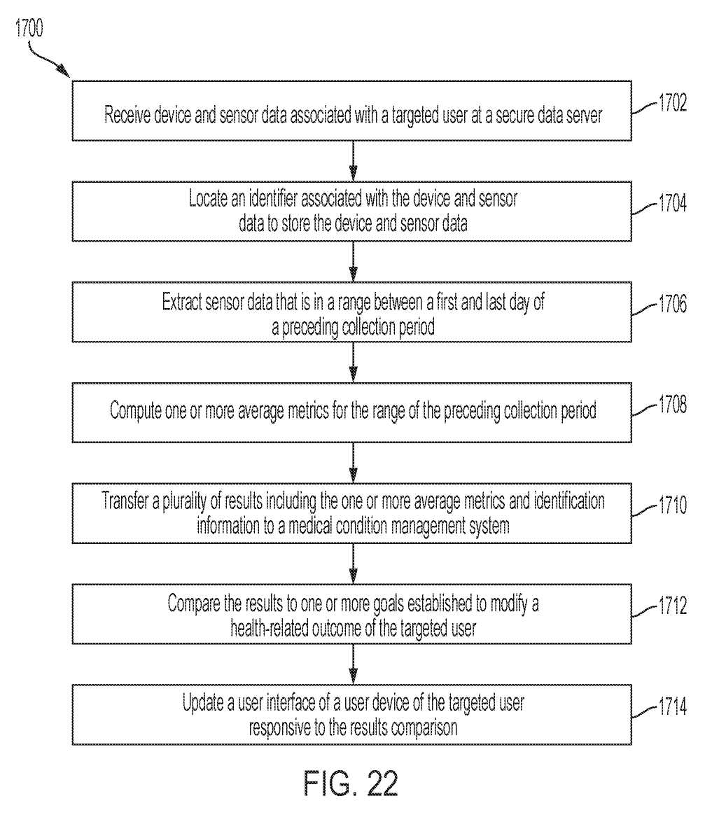

Referring now to FIG. 22 with continued reference to FIGS. 1-21, FIG. 22 is a flow chart illustrating a method 1700, in accordance with an embodiment. The method 1700 may be performed, for example, by the system 100 of FIG. 1. For purposes of explanation, the method 1700 is described primarily with respect to the system 100 of FIG. 1; however, it will be understood that the method 1700 can be performed on other configurations (not depicted).

At block 1702, the first computer system 102 receives a plurality of sensed medical data associated with a targeted user and collected over a first time period. The sensed medical data can include device and sensor data from the data sources 114, including medical devices 120. The first computer system 102 is an example of a secure data server (e.g., server 101) that can execute the secure data collection system 108 to limit access to a plurality of device and sensor data 110. In some instances, uploading of data may be triggered responsive to the targeted user accessing the medical condition management system 148 and viewing one or more time-based goals 502 or in response to an upload request from the medical condition management system 148.

At block 1704, the sensed medical data is stored in a data storage system 104 in combination with a plurality of other medical data associated with a plurality of other users, such as the device and sensor data 110 from users 116. The secure data collection system 108 can locate an identifier associated with one or more of the targeted user, one or more medical devices 120 and sensor data in the data storage system 104 to store the device and sensor data 110. For instance, each insulin pump, CGM, and other such device may have a unique device identifier to assist with tracking device performance and user information. In instances where a same medical device 120 is used by different users over a period of time, such as in a hospital or clinical environment, device-based tracking along with patient-based tracking can assist in tracking general usage trends and personal trends. In some embodiments, the data storage system 104 may be formatted as a data lake, which may store vast amounts of raw data in a native format until it is needed.

At block 1706, the first computer system 102 extracts a subset of the sensed medical data for a second time period from the data storage system 104, where the subset includes a plurality of sensor data associated with a medical device 120 and a plurality of mode data associated with the medical device 120. The first time period can be a variable time period, such as the various data blocks 1602-1610 of FIG. 21, while the second time period may be fixed in predetermined intervals, such as calendar months, as depicted in the example of data blocks 1622, 1624 of FIG. 21. The second period can be a range between a first and last day of a preceding collection period, e.g., October 1-31.

At block 1708, the first computer system 102 computes one or more average metrics for the range of the preceding collection period. For instance, the one or more average metrics can include computing a time-in-range metric based on the sensor data from the subset for the second time period. The one or more average metrics may also compute a total days of sensor data metric, a time in auto-mode metric, and/or other metrics from the subset for the second time period. The time-in-range metric can define an amount of time that the targeted user maintains a health-related outcome within a range bounded by a lower limit and an upper limit, and the total days of sensor data metric can define an amount of time that the targeted user wore a glucose sensor during the second time period. As one example, sensor data for a time between 70 mg/dL (lower limit) and 180 mg/dL (upper limit) can be totaled and divided by the total data available within the second time period to get an average time-in-range for the second time period, which may be expressed as a ratio or percentage. For a medical device 120 supporting a glucose sensor (such as, but not limited to, the Medtronic MiniMed 670G system, or the like), a total number of days the sensor was worn by the user can be computed by determining the total number of data points provided by the user and dividing it by a total amount of number of data points possible in the second time period to express a total days of sensor data metric as a finite number. As another example, the time-in-auto-mode metric can define an amount of time that the targeted user maintains the medical device 120 in an automated mode of dosage control configured to keep the health-related outcome of the targeted user within the range bounded by the lower limit and the upper limit. For a medical device 120 supporting an automated mode of operation, a total time in the automated mode of operation can be computed and divided by a total amount of time in the second time period to express a time-in-auto-mode metric as a ratio or percentage.

At block 1710, a plurality of results including one or more average metrics and identification information can be transferred from the first computer system 102 to the medical condition management system 148 of the second computer system 138. As an example, the time-in-range metric, the total days of sensor data metric, and an identifier associated with the targeted user can be transferred to the second computer system 138 operable to confirm whether the time-in-range metric and the total days of sensor data metric meet at least one goal for the second time period and present one or more results through a user interface for the targeted user. As another example, the time-in-auto-mode metric can be transferred to the second computer system 138 operable to confirm whether the time-in-auto-mode metric meets at least one goal for the second time period and present one or more results through a user interface for the targeted user.

At block 1712, the medical condition management system 148 can compare the results to one or more goals 1406, such as a time-in-range goal and/or a total days of sensor data goal for the second time period (e.g., a month). The one or more goals 1406 can be established to modify a health-related outcome of a targeted user.

At block 1714, the medical condition management system 148 can update a user interface of a user device of the targeted user responsive to the results comparison, such as updating the user interface 500 through the data visualization control 146. The user interface 500 can illustrate a percentage result of the time-in-range metric and/or a numerical result of the total days of sensor data metric graphically as a dashboard, which may also indicate results from multiple time periods, whether an engagement reward was earned, and whether further data uploads are needed, as depicted and described with respect to FIGS. 7-9. The user interface 500 can be provided from the second computer system 138 to a user device of the targeted user, such as the data collection computer 118, mobile device 130, and/or other computing devices 134. As another example, the user interface 550 can illustrate a percentage result of the time-in-range metric and/or the time-in-auto-mode metric graphically as a dashboard, which may also indicate results from multiple time periods, whether a reward was earned, and whether further data uploads are needed, as depicted and described with respect to FIGS. 12-14. The user interface 550 can be provided from the second computer system 138 to a user device of the targeted user, such as the data collection computer 118, mobile device 130, and/or other computing devices 134.

In some instances, data from other sources can be combined in the device and sensor data 110. For instance, the first computer system 102 can receive a plurality of logged entries of medical data, e.g., from other data sources 126. These other sources can include, but are not limited to, medical tests, HbAl c results, medical records, exercise information from devices (such as, but not limited to Fitbits or the like, physiologic sensors, watch computers (similar, but not limited, to an Apple Watch), cellular telephones with physiologic data recording capabilities (such as, but not limited to the iPhone or the like)). The logged entries of medical data can be merged with the sensed medical data. The first computer system 102 can augment the time-in-range metric based on the logged entries of medical data in combination with the sensor data. As a further example, uploading of data can also include receiving a plurality of fitness data based on one or more fitness sensors 128, 132 associated with the targeted user. A fitness summary metric can be determined for the second time period based on the fitness data. For example, the fitness summary metric may be an average weight, an average pulse, an average blood pressure, an average blood oxygen level, an average number of steps, an average period of exercise, and other such values. The fitness summary metric can be transferred to the second computer system 138 for use by the medical condition management system 148.

In some embodiments, the first computer system 102 can analyze the sensed medical data to identify one or more data collection gaps, such as gap 1612. The first computer system 102 can send a data request to a user device associated with the targeted user to provide supplemental data to assist in filling the one or more data collection gaps. For instance, if there are fewer than fifteen days of sensor data, the first computer system 102 may prompt the targeted user to manually enter any log data or data from another source. In some instances, the first computer system 102 may synthesize one or more substitute values to fill in the one or more data collection gaps based on the supplemental data. For instance, if the supplemental data is in a different format or scale than the sensor data, the first computer system 102 may determine a matching format that simulates sensor data based on the supplemental data. The first computer system 102 may augment the time-in-range metric based on the one or more substitute values in combination with the sensor data.

While the above description has described the flow process of FIG. 22 in a particular order, it should be appreciated that unless otherwise specifically required in the attached claims that the ordering of the steps may be varied.

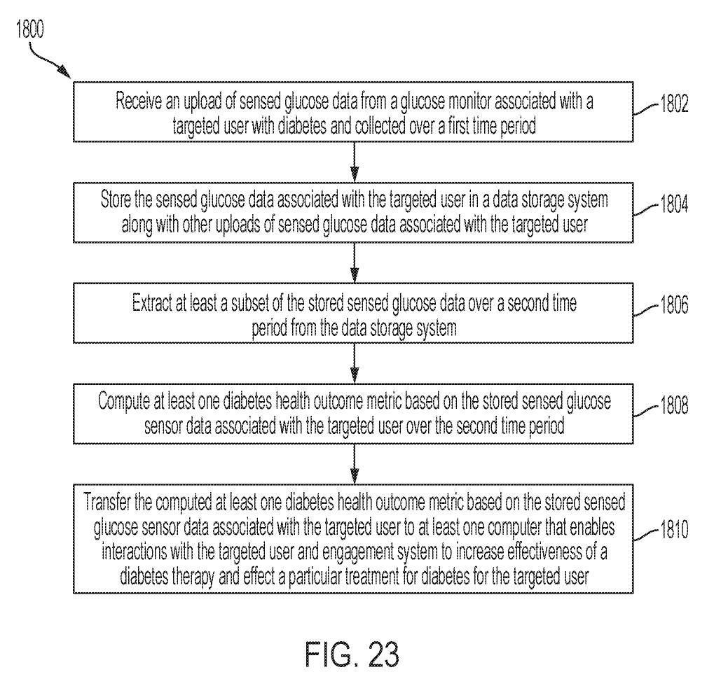

Referring now to FIG. 23 with continued reference to FIGS. 1-22, FIG. 23 is a flow chart illustrating a method 1800, in accordance with an embodiment. The method 1800 may be performed, for example, by the system 100 of FIG. 1. For purposes of explanation, the method 1800 is described primarily with respect to the system 100 of FIG. 1; however, it will be understood that the method 1800 can be performed on other configurations (not depicted).

As previously described, an engagement system 100 can include at least one computer to provide interactions with a targeted user with diabetes, such as one or more users 116. The at least one computer can be selected from a group including a personal computer, laptop computer, cloud computing resource, or an ultra-book computer, such as the data collection computer 118 and/or second computer system 138. Further, the at least one computer can be selected from the group including a cellular telephone, a smartphone, a wearable computing device, a personal digital assistant or a tablet, such as mobile device 130 and/or other computing devices 134. The engagement system 100 can increase effectiveness of a diabetes therapy and effect a particular treatment for diabetes for the targeted user by suggesting actions or rewarding the targeted user for taking actions to improve diabetes health outcomes measured by at least one diabetes health outcome metric that meets a success criteria in response to the interactions or taken actions by the targeted user. The engagement system 100 can also include a server 101 communicatively coupled with the at least one computer, such as the computer system 138 and/or other computing devices. The server 101 can include or access a data storage system and a processing system configured to perform a plurality of operations, such as the operation of blocks 1802, 1804, 1806, 1808, and 1810 of method 1800, as well as additional operations. For example, the server 101 can include the first computer system 102 with data storage system 104 and processing system 106. The server 101 may also include or interface with the second computer system 138 and data storage system 142.

At block 1802, the server 101 can receive an upload of sensed glucose data from a glucose monitor associated with the targeted user and collected over a first time period. The sensed glucose data can be collected from one or more medical devices 120, such as sensor system 124. The sensed glucose data can be continuous sensor data from a monitored continuous sensor that provides sensed glucose data at least once every 20 minutes over at least 12 hours or includes on demand uploads of the continuous sensor data from the targeted user. A time length of 20 minutes is provided as an example. It will be understood that the time length of 20 minutes can vary, for instance, as a shorter time (e.g., 5, 10, 15 minutes, etc.) or as a longer time (e.g., 25, 30, 35, 40, 45 minutes to an hour or more, etc.). Further, the duration of 12 hours is merely an example, and other durations can be implemented in various embodiments. For instance, the duration can be shorter, such as 6 hours, or longer, such as 1, 2, 3, 4, 5, 6, 7 days or more. On demand uploads can be provided at a user-defined interval or in response to a user-initiated request, such as using the most recent sensor data. The engagement system can be configured to update records on demand as well. At block 1804, the sensed glucose data associated with the targeted user can be stored in the data storage system 104 along with other uploads of sensed glucose data associated with the targeted user. At block 1806, at least a subset of the stored sensed glucose data over a second time period can be extracted from the data storage system 104. At block 1808, at least one diabetes health outcome metric can be computed based on the stored sensed glucose sensor data associated with the targeted user over the second time period.

At block 1810, the computed at least one diabetes health outcome metric based on the stored sensed glucose sensor data associated with the targeted user can be transferred to the at least one computer. The at least one computer can interact with the targeted user based on the transferred at least one diabetes health outcome metric with further interactions, suggested actions or rewards to the targeted user based on whether the computed at least one diabetes health outcome metric met the success criteria.

The interactions on the at least one computer with the targeted user can include at least one of: asking questions, logging results, providing articles related to the diabetes therapy, providing glucose sensor data graphs, providing analysis of glucose sensor data, providing recommendations of diabetes therapy suggested actions, displaying the computed at least one diabetes health outcome metric, notifying the targeted user on whether success criteria of the computed at least one diabetes health outcome metric has been met, or notifying the targeted user of an reward. The rewards can include at least one of: points, points redeemable for merchandise, a credit on an account, a credit applied to a co-pay, or an achievement badge.

The computed at least one diabetes health outcome metric can be a time-in-range for the subset of stored sensed glucose data associated with the targeted user, and the time-in-range can be the time that the subset of the stored sensed glucose data is not in a low sensor glucose state. For example, the low sensor glucose state can be below 60 mg/dl. The time-in-range can be the time that the subset of the stored sensed glucose data is also not in a high sensor glucose state. For example, the high sensor glucose state can be above 180 mg/dl. Further, the time-in-range can be between the low sensor glucose state and the high sensor glucose state. The time-in-range can be shown as a percent of the second time period. As one example, the success criteria for the time-in-range can be at or above 75%. As another example, the success criteria for the time-in-range can be at or above 80%.

While the above description has described the flow process of FIG. 23 in a particular order, it should be appreciated that unless otherwise specifically required in the attached claims that the ordering of the steps may be varied.

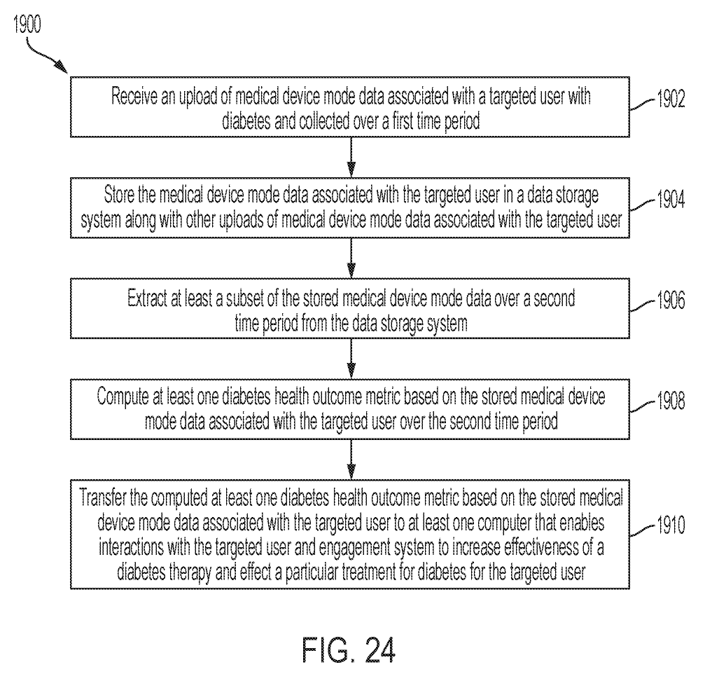

Referring now to FIG. 24 with continued reference to FIGS. 1-23, FIG. 24 is a flow chart illustrating a method 1900, in accordance with an embodiment. The method 1900 may be performed, for example, by the system 100 of FIG. 1. For purposes of explanation, the method 1900 is described primarily with respect to the system 100 of FIG. 1; however, it will be understood that the method 1900 can be performed on other configurations (not depicted).

At block 1902, a server 101 communicatively coupled with at least one computer can receive an upload of medical device mode data associated with a targeted user with diabetes and collected over a first time period. The medical device mode data can be for a device to control diabetes in a closed-loop mode for at least a portion of the first time period. At block 1904, the medical device mode data associated with the targeted user can be stored in a data storage system 104 of the server 101 along with other uploads of medical device mode data associated with the targeted user. At block 1906, at least a subset of the stored medical device mode data over a second time period can be extracted from the data storage system 104. At block 1908, at least one diabetes health outcome metric can be computed based on the stored medical device mode data associated with the targeted user over the second time period.

At block 1910, the computed at least one diabetes health outcome metric based on the stored medical device mode data associated with the targeted user can be transferred to the at least one computer. The at least one computer can interact with the targeted user based on the transferred at least one diabetes health outcome metric with interactions, suggested actions or rewards to the targeted user based on whether the computed at least one diabetes health outcome metric met a success criteria to increase effectiveness of a diabetes therapy and effect a particular treatment for diabetes for the targeted user by suggesting actions or rewarding the targeted user for taking actions to improve diabetes health outcomes measured by the computed at least one diabetes health outcome metric that meets the success criteria in response to the interactions or taken actions by the targeted user.

In embodiments, the interactions on the at least one computer with the targeted user can include at least one of: asking questions, logging results, providing articles related to the diabetes therapy, providing medical device mode data graphs, providing analysis of medical device mode data, providing recommendations of diabetes therapy suggested actions, displaying the computed at least one diabetes health outcome metric, notifying the targeted user on whether success criteria of the computed at least one diabetes health outcome metric has been met, or notifying the targeted user of an reward. The rewards can include at least one of: points, points redeemable for merchandise, a credit on an account, a credit applied to a co-pay, or an achievement badge.

The computed at least one diabetes health outcome metric can be a time-in-closed-loop mode for the subset of the stored medical device mode data associated with the targeted user, and the time-in-closed-loop mode can be the time that the subset of the stored medical device mode data is not in open-loop mode. The time-in-closed-loop mode can be based on data obtained from a system including an insulin infusion device and a glucose sensor in communication with the insulin infusion device, for example. The time-in-closed-loop mode can be shown as a percent of the second time period. As one example, the success criteria for the time-in-closed-loop mode can be at or above 75%. As another example, the success criteria for the time-in-closed-loop mode can be at or above 80%.

While the above description has described the flow process of FIG. 24 in a particular order, it should be appreciated that unless otherwise specifically required in the attached claims that the ordering of the steps may be varied.

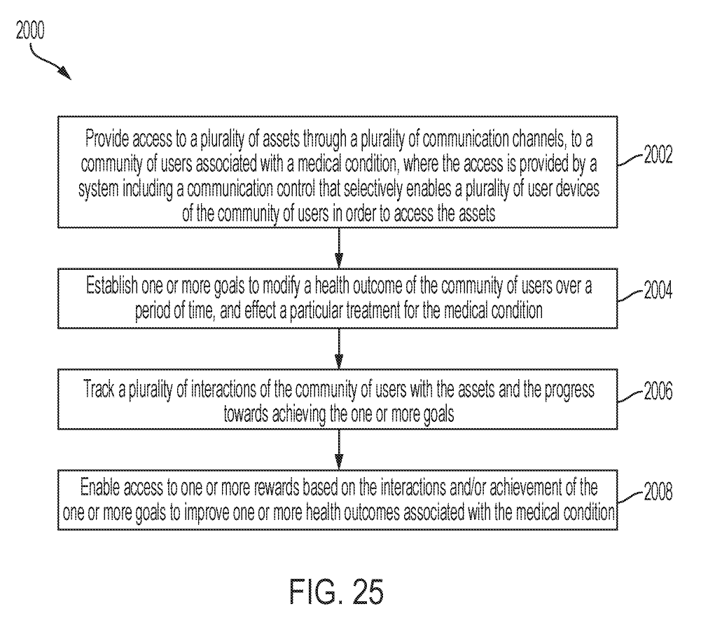

Referring now to FIG. 25 with continued reference to FIGS. 1-24, FIG. 25 is a flow chart illustrating a method 2000, in accordance with an embodiment. The method 2000 may be performed, for example, by the system 100 of FIG. 1. For purposes of explanation, the method 2000 is described primarily with respect to the system 100 of FIG. 1; however, it will be understood that the method 2000 can be performed on other configurations (not depicted).

At block 2002, the system 100 provides access to a plurality of assets 1410 through a plurality of communication channels, to a community of users 116 associated with a medical condition. For example, a communication control 1408 of the medical condition management system 148 can provide the users 116 with access to the assets 1410 and may send notifications or trigger events to user devices of the user 116, such as the data collection computer 118, mobile device 130, and/or other computing devices 134, to perform one or more activities 1502. The communication control 1408 can selectively enable a plurality of user devices of the community of users in order to access the assets 1410. The assets 1410 can include one or more embedded assets 1412 locally accessible by the second computer system 138 and/or one or more linked assets 1414 accessible through a network 136 operably coupled to the second computer system 138, such as the linked computer systems 150A-150N.

At block 2004, the system 100 can establish one or more goals 1406 to modify a health outcome of the community of users 116 over a period of time and effect a particular treatment for the medical condition. The one or more goals 1406 can include a time-in-range metric that defines an amount of time that a user of the community of users 116 maintains the health-related outcome within a range bounded by a lower limit and an upper limit. The one or more goals 1406 may also include a time-in-auto-mode metric that defines an amount of time that the user maintains a medical device 120 in an automated mode of dosage control configured to keep the health-related outcome of the user within the range bounded by the lower limit and the upper limit. Alternatively, other metrics can be used, such as a total days of sensor data metric that defines an amount of time that the user wore a glucose sensor. The total days of sensor data metric can be from continuous sensor data from a monitored continuous sensor that provides sensed glucose data at least once every 20 minutes over at least 12 hours or includes on demand uploads of the continuous sensor data from the user, for example. A time length of 20 minutes is provided as an example. It will be understood that the time length of 20 minutes can vary, for instance, as a shorter time (e.g., 5, 10, 15 minutes, etc.) or as a longer time (e.g., 25, 30, 35, 40, 45 minutes to an hour or more, etc.). Further, the duration of 12 hours is merely an example, and other durations can be implemented in various embodiments. For instance, the duration can be shorter, such as 6 hours, or longer, such as 1, 2, 3, 4, 5, 6, 7 days or more. On demand uploads can be provided at a user-defined interval or in response to a user-initiated request, such as using the most recent sensor data. The engagement system can be configured to update records on demand as well. A process control 1402 can vary the one or more goals 1406 to establish a plurality of challenges to the community of users 116, where the challenges are modified over a period of time to increase a number of users 116 trending towards achieving the health-related outcome. The process control 1402 can apply one or more models 1404 to sequence a plurality of activities 1502, feedback, and reward points in the challenges. The activities 1502 can include one or more of: data updates, educational resource access, surveys, video content, medication delivery scheduling, social media access, and interactions between the community of users 116. A personalization controller 1540 can make one or more personalized adjustments to the challenges for one or more users 116 of the community of users 116. The goals may also be preventative for users who are trending toward a medical condition or may desire to prevent a medical condition.

At block 2006, the system 100 can track a plurality of interactions of the community of users 116 with the assets 1410 and the progress towards achieving the one or more goals 1406. At block 2008, the system 100 can enable access to one or more engagement rewards based on the interactions and/or achievement of the one or more goals 1406 to improve one or more health outcomes associated with the medical condition.

While the above description has described the flow process of FIG. 25 in a particular order, it should be appreciated that unless otherwise specifically required in the attached claims that the ordering of the steps may be varied.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors (e.g., on processing systems 106, 144, 152A-152N of FIG. 1), such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method comprising:
providing access to a plurality of assets through a plurality of communication channels, to a community of users associated with a medical condition, wherein the access is provided by a system comprising a communication control that selectively enables a plurality of user devices of the community of users in order to access the assets;
establishing one or more goals to modify a health-related outcome of the community of users over a period of time, and effecting a particular treatment for the medical condition, wherein the one or more goals comprise a time-in-range metric that defines an amount of time that a user of the community of users maintains the health-related outcome within a range bounded by a lower limit and an upper limit, wherein the one or more goals comprise a total days of sensor data metric that defines an amount of time that the user wore a glucose sensor, and wherein the total days of sensor data metric is from continuous sensor data from a monitored continuous sensor that provides sensed glucose data at least once every 20 minutes over at least 12 hours or includes on demand uploads of the continuous sensor data from the user;
tracking a plurality of interactions of the community of users with the assets and the progress towards achieving the one or more goals; and
enabling access to one or more engagement rewards based on the interactions and/or achievement of the one or more goals to improve one or more health outcomes associated with the medical condition.

2. The method of claim 1, wherein the assets comprise one or more embedded assets locally accessible by a computer system and one or more linked assets accessible through a network operably coupled to the computer system.

3. The method of claim 1, further comprising:
varying the one or more goals to establish a plurality of challenges to the community of users, wherein the challenges are modified over time to increase a number of users trending towards achieving the health-related outcome.

4. The method of claim 3, further comprising:
applying one or more models to sequence a plurality of activities, feedback, and engagement reward points in the challenges that encourage the community of users to achieve higher levels of a plurality of health tiers.

5. The method of claim 4, wherein the activities comprise one or more of: data updates, educational resource access, surveys, video content, medication delivery scheduling, social media access, and interactions between the community of users.

6. The method of claim 4, further comprising:
making one or more personalized adjustments to the challenges for one or more users of the community of users; and
enabling one or more interactions between a plurality of users and/or coaches through the communication channels targeting one or more health outcome improvements.

7. A system comprising:
a data storage system; and
a computer system communicatively coupled to the data storage system, the computer system comprising a processing system configured to perform a plurality of operations comprising:
providing access to a plurality of assets through a plurality of communication channels, to a community of users associated with a medical condition, wherein the access is provided through a communication control of the system that selectively enables a plurality of user devices of the community of users in order to access the assets;
establishing one or more goals to modify a health outcome of the community of users over a period of time, and effecting a particular treatment for the medical condition, wherein the one or more goals comprise a time-in-range metric that defines an amount of time that a user of the community of users maintains the health-related outcome within a range bounded by a lower limit and an upper limit, wherein the one or more goals comprise a total days of sensor data metric that defines an amount of time that the user wore a glucose sensor, and wherein the total days of sensor data metric is from continuous sensor data from a monitored continuous sensor that provides sensed glucose data at least once every 20 minutes over at least 12 hours or includes on demand uploads of the continuous sensor data from the user;
tracking a plurality of interactions of the community of users with the assets and the progress towards achieving the one or more goals; and
enabling access to one or more engagement rewards based on the interactions and/or achievement of the one or more goals to improve one or more health outcomes associated with the medical condition.

8. The system of claim 7, wherein the assets comprise one or more embedded assets locally accessible by the computer system and one or more linked assets accessible through a network operably coupled to the computer system.

9. The system of claim 7, wherein the computer system is further configured to perform operations comprising:
varying the one or more goals to establish a plurality of challenges to the community of users, wherein the challenges are modified over time to increase a number of users trending towards achieving the health-related outcome; and
applying one or more models to sequence a plurality of activities, feedback, and engagement reward points in the challenges that encourage the community of users to achieve higher levels of a plurality of health tiers, wherein the activities comprise one or more of: data updates, educational resource access, surveys, video content, medication delivery scheduling, social media access, and interactions between the community of users.

10. The system of claim 9, wherein the computer system is further configured to perform operations comprising:
making one or more personalized adjustments to the challenges for one or more users of the community of users; and
enabling one or more interactions between a plurality of users and/or coaches through the communication channels targeting one or more health outcome improvements.

11. A computer program product comprising a storage medium embodied with computer program instructions that when executed by a computer system cause the computer system to implement:
providing access to a plurality of assets through a plurality of communication channels, to a community of users associated with a medical condition, wherein the access is provided through a communication control of the computer system that selectively enables a plurality of user devices of the community of users in order to access the assets;
establishing one or more goals to modify a health outcome of the community of users over a period of time, and effecting a particular treatment for the medical condition, wherein the one or more goals comprise a time-in-range metric that defines an amount of time that a user of the community of users maintains the health-related outcome within a range bounded by a lower limit and an upper limit, and wherein a total days of sensor data metric that defines an amount of time that the user maintains a medical device in an automated mode of dosage control configured to keep the health-related outcome of the user within the range bounded by the lower limit and the upper limit, wherein the one or more goals comprise a total days of sensor data metric that defines an amount of time that the user wore a glucose sensor, and wherein the total days of sensor data metric is from continuous sensor data from a monitored continuous sensor that provides sensed glucose data at least once every 20 minutes over at least 12 hours or includes on demand uploads of the continuous sensor data from the user;
tracking a plurality of interactions of the community of users with the assets and the progress towards achieving the one or more goals; and
enabling access to one or more engagement rewards based on the interactions and/or achievement of the one or more goals to improve one or more health outcomes associated with the medical condition.

12. The computer program product of claim 11, wherein the assets comprise one or more embedded assets locally accessible by the computer system and one or more linked assets accessible through a network operably coupled to the computer system.

13. The computer program product of claim 11, further comprising computer program instructions that when executed by the computer system cause the computer system to implement:
varying the one or more goals to establish a plurality of challenges to the community of users, wherein the challenges are modified over time to increase a number of users trending towards achieving the health-related outcome.

14. The computer program product of claim 13, further comprising computer program instructions that when executed by the computer system cause the computer system to implement:
applying one or more models to sequence a plurality of activities, feedback, and engagement reward points in the challenges that encourage the community of users to achieve higher levels of a plurality of health tiers.

15. The computer program product of claim 14, wherein the activities comprise one or more of: data updates, educational resource access, surveys, video content, medication delivery scheduling, social media access, and interactions between the community of users.

16. The computer program product of claim 14, further comprising computer program instructions that when executed by the computer system cause the computer system to implement:
making one or more personalized adjustments to the challenges for one or more users of the community of users; and
enabling one or more interactions between a plurality of users and/or coaches through the communication channels targeting one or more health outcome improvements.
